(19) United States
(12) Patent Application Publication
(10) Pub. No.: US 2004/0003419 A1
(43) Pub. Date: Jan. 1, 2004

(54) Novel taste receptors in Drosophila

(75) Inventors: John R. Carlson, North Haven, CT (US); Peter J. Clyne, San Francisco, CA (US); Coral G. Warr, New Haven, CT (US)

Correspondence Address: MORGAN LEWIS & BOCKIUS LLP, 1111 PENNSYLVANIA AVENUE NW, WASHINGTON, DC, 20004, US

(73) Assignee: Yale University

(21) Appl. No.: 10/447,328

(22) Filed: May 29, 2003

Related U.S. Application Data
(63) Continuation of application No. 09/593,519, filed on Jun. 14, 2000, now abandoned.
(60) Provisional application No. 60/181,704, filed on Feb. 10, 2000.
(60) Provisional application No. 60/138,668, filed on Jun. 14, 1999.

(51) Int. Cl. A01K 67/033; C07H 21/04; C12N 5/06; C07K 14/705; C12P 21/02

(52) U.S. Cl. 800/8; 435/69.1; 435/348; 435/320.1; 530/350; 536/23.5

(57) ABSTRACT

The present invention provides nucleic acids and amino acids for novel gustatory receptors as well as methods for identifying gustatory receptors. More specifically, the present invention provides nucleic acids and amino acids for novel gustatory receptors in Drosophila as well as methods of using the provided nucleic acids and amino acids. In addition, this invention provides methods of identifying ligands which bind to the novel gustatory receptors as well as a variety of methods for using the receptors and ligands so identified.

### RELATED APPLICATIONS

This application claims priority to U.S. provisional patent application No. 60/181,704 filed Feb. 10, 2000 and U.S. provisional patent application No. 60/138,668 filed Jun. 14, 1999 both applications herein incorporated by reference in their entirety.

### U.S. GOVERNMENT SUPPORT

This work was supported by a grant from the National Institutes of Health (DC-02174).

### FIELD OF THE INVENTION

This invention pertains to novel taste receptors and to methods of using such receptors. More particularly, this invention pertains to the nucleic acids and amino acids of novel taste receptors in Drosophila and to methods of using such nucleic acids and amino acids.

### BACKGROUND OF THE INVENTION

Studies in insect gustation have a long history in general physiology. Much more emphasis has been placed on the physiological characteristics of the sensory cells than on the central cellular mechanisms of taste processing (Mitchell et al., (1999) Microsc. Res. Tech. 47, 401-415). For a survey review of the organization of the olfactory and gustatory systems in Drosophila, see Stocker, (1994) Cell Tissue Res. 275, 3-26.

Both male and female Drosophila flies possess gustatory receptors on their legs, but males possess more of these receptors than females (Possidente et al., (1989) Dev. Biol. 132, 448-457). The labellar hairs of larger flies are not only sensitive to a variety of simple and compound sugars (Dethier, (1955) Q. Rev. Biol. 30, 348), but also to a wide variety of other molecules, such as amino acids (Shiraishi & Kuwabara, (1970) J. Gen. Physiol. 56, 768).

Behavioral studies have shown that Drosophila are sensitive to quinine (Tompkins et al., (1979) Proc. Natl. Acad. Sci. USA 76, 884), which is perceived by humans as bitter, and other insects have been shown to be sensitive to an array of structurally diverse bitter compounds. Moreover, an individual insect taste receptor cell can respond to a broad range of structurally heterogeneous alkaloids and other bitter molecules (Glendinning & Hills, (1997) J. Neurophysiol. 78, 734; Chapman et al., (1991) J. Exp. Biol. 158, 241).

Little is known about the molecular mechanisms of taste perception in animals, particularly the initial events of taste signaling. Several genes known to affect the formation of gustatory sensilla or alter the feeding behavior of Drosophila have been identified (Singh, (1997) Microsc. Res. Tech. 3, 547-563). For example, a mutation in the malvolio (mvl) gene affects taste behavior in Drosophila melanogaster (Orgad et al., (1998) J. Exp. Biol. 201, 115-120). Also, scalloped (sd) mutants show defects in response to a number of taste stimuli (Inamdar et al., (1993) J. Neurogenet. 9, 123-139). Flies containing different Shaker alleles exhibit a variety of defects in their gustatory response to sucrose, NaCl and KCl (Balakrishnan et al., (1991) J. Exp. Biol. 157, 161-181).

Although two putative mammalian taste receptors have recently been described (Hoon et al., (1999) Cell 96, 541), remarkably little is understood in general about taste receptors across species.

In the present invention, a large and diverse family of seven transmembrane domain proteins was identified from the Drosophila genome database with a computer algorithm that identifies proteins on the basis of structure. Eighteen of nineteen genes examined were expressed in the Drosophila labellum, a gustatory organ of the proboscis. Expression was not detected in a variety of other tissues. The genes were not expressed in the labellum of a Drosophila mutant, pox-neuro 70, in which taste neurons are eliminated. Tissue specificity of expression of these genes, along with their structural similarity, supports the possibility that the family encodes a large and divergent family of taste receptors.

### SUMMARY OF THE INVENTION

This invention provides isolated nucleic acid molecules including the following:

(a) isolated nucleic acid molecules that encode the amino acid sequences of Drosophila Gustatory Receptor proteins; (b) isolated nucleic acid molecules that encode protein fragments of at least six amino acids of a Drosophila Gustatory Receptor proteins; and (c) isolated nucleic acid molecules which hybridize to nucleic acid molecules which include nucleotide sequences encoding Drosophila Gustatory Receptor proteins under conditions of sufficient stringency to produce a clear signal.

This invention also provides such isolated nucleic acid molecules wherein the nucleic acids include at least one exon-intron boundary located in one of the following positions: (a) the nucleotides encoding the amino acids which include the third extracellular loop of a Drosophila Gustatory Receptor protein; and (b) the nucleotides encoding the amino acids which include the seventh transmembrane domain of a Drosophila Gustatory Receptor protein.

This invention further provides such isolated nucleic acid molecules which have the nucleic acid sequence of one of the following sequences: SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91.

This invention also provides such isolated nucleic acid molecules operably linked to one or more expression control elements.

This invention further provides vectors which include any of the aforementioned nucleic acid molecules and host cells which include such vectors.

This invention also provides host cells transformed so as to contain any of the aforementioned nucleic acid molecules, wherein such host cells can be either prokaryotic host cells or eukaryotic host cells.

This invention also provides methods for producing proteins or protein fragments wherein the methods include transforming host cells with any of the aforementioned nucleic acids under conditions in which the protein or protein fragment encoded by said nucleic acid molecule is expressed. This invention also provides such methods wherein the host cells are either prokaryotic host cells or eukaryotic host cells. This invention further provides isolated proteins or protein fragments produced by such methods.

This invention provides isolated proteins or protein fragments which include: (a) isolated proteins encoded by one of the following amino acid sequences: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92; (b) isolated protein fragments which include at least six amino acids of any of the following sequences: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92; (c) isolated proteins which include conservative amino acid substitutions of any of the following sequences: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92; and (d) naturally occurring amino acid sequence variants of any of the following sequences: SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92.

The present invention further provides such isolated proteins or protein fragments which include at least one of the following conserved amino acids: Serine in the amino terminal domain; Phenylalanine in the first transmembrane domain; Arginine in the first extracellular loop; Leucine in the fourth transmembrane domain; Leucine in the third transmembrane domain; Glycine in the fifth transmembrane domain; Tyrosine in the fifth transmembrane domain; Leucine in the third extracellular loop; Phenylalanine in the third extracellular loop; Alanine in the seventh transmembrane domain; Glycine in the seventh transmembrane domain; Leucine in the seventh transmembrane domain; Aspartate in the seventh transmembrane domain; Alanine in the seventh transmembrane domain; Threonine in the seventh transmembrane domain; Tyrosine in the seventh transmembrane domain; Valine in the seventh transmembrane domain; Glutamine in the carboxy terminal domain; and Phenylalanine in the carboxy terminal domain.

The present invention also provides isolated antibodies that bind to any of the aforementioned polypeptides. The present invention also provides such antibodies which are either monoclonal antibodies or polyclonal antibodies.

This invention also provides methods of identifying agents which modulate the expression of any of the aforementioned proteins or protein fragments by: (a) exposing cells which express the proteins or protein fragments to the agents; and (b) determining whether the agent modulates expression of said proteins or protein fragments, thereby identifying agents which modulate the expression of the proteins or protein fragments.

The present invention also provides methods of identifying agents which modulate the activity of any of the aforementioned proteins or protein fragments by: (a) exposing cells which express the proteins or protein fragments to the agents; and (b) determining whether the agents modulate the activity of said proteins or protein fragments, thereby identifying agents which modulate the activity of the proteins or protein fragments.

The present invention also provides such methods where the agent modulates at least one activity of the proteins or protein fragments.

This invention provides methods of identifying agents which modulate the transcription of any of the aforementioned nucleic acid molecules by: (a) exposing cells which transcribe the nucleic acids to the agents; and (b) determining whether the agents modulate transcription of said nucleic acids, thereby identifying agents which modulate the transcription of the nucleic acid.

This invention further provides methods of identifying binding partners for the aforementioned proteins or protein fragments by: (a) exposing said proteins or protein fragments to potential binding partners; and (b) determining if the potential binding partners bind to said proteins or protein fragments, thereby identifying binding partners for the proteins or protein fragments.

The present invention also provides methods of modulating the expression of nucleic acids encoding the aforementioned proteins or protein fragments by administering an effective amount of agents which modulate the expression of the nucleic acids encoding the proteins or protein fragments.

This invention also provides methods of modulating at least one activity of the aforementioned proteins or protein fragments by administering an effective amount of the agents which modulate at least one activity of the proteins or protein fragments.

This invention provides methods of identifying novel gustatory receptor genes by:(a) selecting candidate gustatory receptor genes by screening nucleic acid databases using an algorithm trained to identify seven transmembrane receptors genes; (b) screening said selected candidate gustatory receptor genes by identifying nucleic acid sequences with conserved amino acid residues and intron-exon boundaries common to gustatory receptors, and having open reading frames of sufficient size so as to encode a seven transmembrane receptor; and (c) identifying the novel gustatory receptor genes and measuring the expression of gustatory receptor genes wherein the detection of expression confirms said candidate gustatory genes as gustatory genes.

This invention also provides methods of identifying novel gustatory receptor genes by: (a) selecting candidate gustatory receptor genes by screening nucleic acid databases for nucleic acid sequences with sufficient homology to at least one known gustatory receptor gene; (b) screening said selected candidate gustatory receptor genes by identifying nucleic acids with conserved amino acid residues and intron-exon boundaries common to gustatory receptors, and having open reading frames of sufficient size so as to encode a seven transmembrane receptor; and (c) identifying the novel gustatory receptor genes and measuring the expression of gustatory receptor genes wherein the detection of expression confirms said candidate gustatory genes as gustatory genes.

The present invention also provides transgenic insects modified to contain any of the aforementioned nucleic acid molecules.

This invention also provides such transgenic insects, wherein the nucleic acid molecules contain mutations that alter expression of the encoded proteins.

### BRIEF DESCRIPTION OF THE DRAWINGS

FIG. 1 Amino acid sequence alignment of nineteen GR proteins. Single-letter abbreviations for the amino acid residues are as follows: A, Ala; C, Cys; D, Asp; E, Glu; F, Phe; G, Gly; H, His; I, Ile; K, Lys; L, Leu; M, Met; N, Asn; P, Pro; Q, Gln; R, Arg; S, Ser; T, Thr; V, Val; W, Trp; and Y, Tyr. Letters following protein designations identify alternative splicing products of individual genes. Residues conserved in >50% of the predicted proteins are shaded. The approximate locations of the seven predicted transmembrane domains are indicated. Intron-exon boundaries are shown with vertical lines. The sequences shown are the first 19 full-length proteins identified. All DNA sequences are from the Berkeley Drosophila Genome Project (BDGP) database. See the Examples for a complete description.

FIG. 2 Representative hydropathy plots of GR proteins. Hydrophobic peaks predicted by Kyte-Doolittle analysis appear above the center lines. The approximate positions of the seven putative transmembrane domains are indicated above the first hydropathy plot. Similar plots were obtained for all of the GR proteins.

FIG. 3 Genomic organization of the 39D.2 and 23A.1 loci. In the 39D.2 locus, the gray boxes labeled a through d represent four large 5′ exons, each of which can be spliced individually to the three 3′ exons (indicated in black) to produce alternative transcripts encoding four different proteins. All the exons of the 39D.2 locus are located in an intron of another gene, which is in the opposite orientation and whose exons are represented by white boxes. This other gene appears to encode a basic helix-loop-helix transcription factor expressed during embryogenesis. In the 23A.1 locus, the gray boxes labeled a and b represent two alternative large 5′ exons, either of which can be spliced to the two small 3′ exons (indicated in black) to produce transcripts encoding two different proteins.

FIG. 4 Tissue specificity of expression of 32D.1 in the labellum. Shown is a gel photograph of an RT-PCR experiment with primers spanning an intron in 32D.1. The size of the predicted PCR product from cDNA is 372 base pairs; any remaining genomic DNA would generate a product of 559 base pairs. A cDNA band is observed in the labellum lane only. In addition, 32D. 1 is not expressed in the labellum of the poxn 70 mutant. Positive controls are described in the Examples.

The amount of each tissue used to prepare cDNA was that determined to give approximately the same signal with a pair of positive control primers, CGGATCCCTATGTCAAGGTG (SEQ ID NO: 93) and GAAGAGCTTCGTGCTGGTCT (SEQ ID NO: 94), representing the Drosophila synaptotagmin gene (Perin et al., (1991) J. Biol. Chem. 266, 615). Specifically, the amount of tissue used in each cDNA preparation was as follows: fifty labella, five heads from which taste organs (the labellum, the LSO, the dorsal cibarial sense organ, and the ventral cibarial sense organ) had been surgically removed, twenty thoraces, twenty abdomens, two-hundred legs, and twenty anterior wing margins (the portion of the wing containing chemosensory sensilla).

Complementary DNA preparation and PCR were performed as in Clyne et al., (1999) Neuron 22, 327-338. For all genes, primer pairs (Available as supplementary material at www.sciencemag.org/feature/data/1046815.shl) that span introns were used to distinguish bands amplified from cDNA from those amplified from any remaining genomic DNA. All negative results were confirmed by testing at least one additional primer pair.

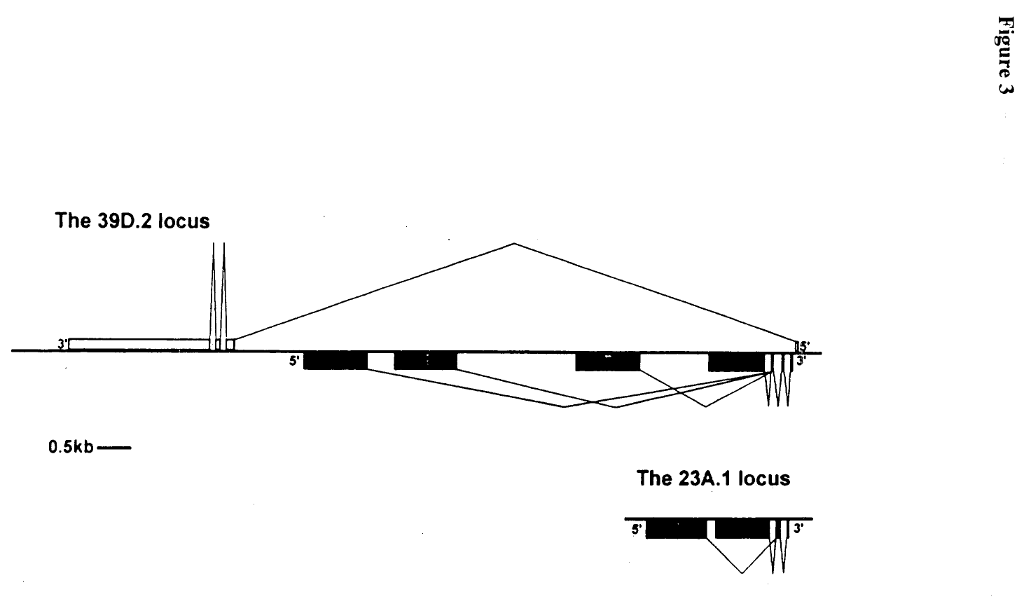

FIG. 5 GR gene expression in microdissected labral sense organs (LSOs). The shaded areas show the four major taste organs of the Drosophila head: the LSO, the dorsal cibarial sense organ (DCSO), the ventral cibarial sense organ (VCSO), and the labellum. The gel track shows an amplification product from RNA extracted from fifty LSOs, amplified with primers N23A.3J and N23A.2D from two exons of gene 23A.1. Specifically, one primer is from the large exon 23A.1a (FIG. 3), and the other is from the First common exon at the 3′ end. The amplification product is 430 base pairs, which is the expected length for a cDNA product; any remaining genomic DNA would generate a product of 1598 base pairs. The primer pair did not amplify a product from non-gustatory tissue (see Examples, Table 1). The following transcripts were detected in the LSO: 22B.1, 23A.1a, 23A.1b, 32D.1, 39D.2c, 43C.1, and 58A.2.

### DETAILED DESCRIPTION OF THE PREFERRED EMBODIMENT

I. Specific Embodiments

A. Drosophila Gustatory Receptor Proteins

The present invention provides a family of isolated proteins, allelic variants of the proteins, and conservative amino acid substitutions of the proteins. As used herein, protein or polypeptide refers to any one of the proteins that has the amino acid sequence depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92. The invention also includes naturally occurring allelic variants and proteins that have a slightly different amino acid sequence than that specifically recited above. Allelic variants, though possessing a slightly different amino acid sequence than those recited above, will still have the same or similar biological functions associated with any of the amino acid proteins.

As used herein, the family of proteins related to any one of the amino acid sequences depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92 refers to proteins that have been isolated from organisms in addition to Drosophila. The methods used to identify and isolate other members of the family of proteins related to these amino acid proteins are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include conservative amino acid substitution variants (i.e., conservative) of the proteins herein described. As used herein, a conservative variant refers to at least one alteration in the amino acid sequence that does not adversely affect the biological functions of the protein. A substitution, insertion or deletion is said to adversely affect the protein when the altered sequence prevents or disrupts a biological function associated with the protein. For example, the overall charge, structure or hydrophobic-hydrophilic properties of the protein can be altered without adversely affecting a biological activity. Accordingly, the amino acid sequence can often be altered, for example to render the peptide more hydrophobic or hydrophilic, without adversely affecting the biological activities of the protein.

Ordinarily, the allelic variants, the conservative substitution variants, and the members of the protein family, will have an amino acid sequence having at least 10% amino acid sequence identity with the sequences set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84 or 86 more preferably at least 35%, even more preferably at least 40% and most preferably at least 45%. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N-terminal, C-terminal or internal extensions, deletions or insertions into the peptide sequence shall not be construed as affecting homology.

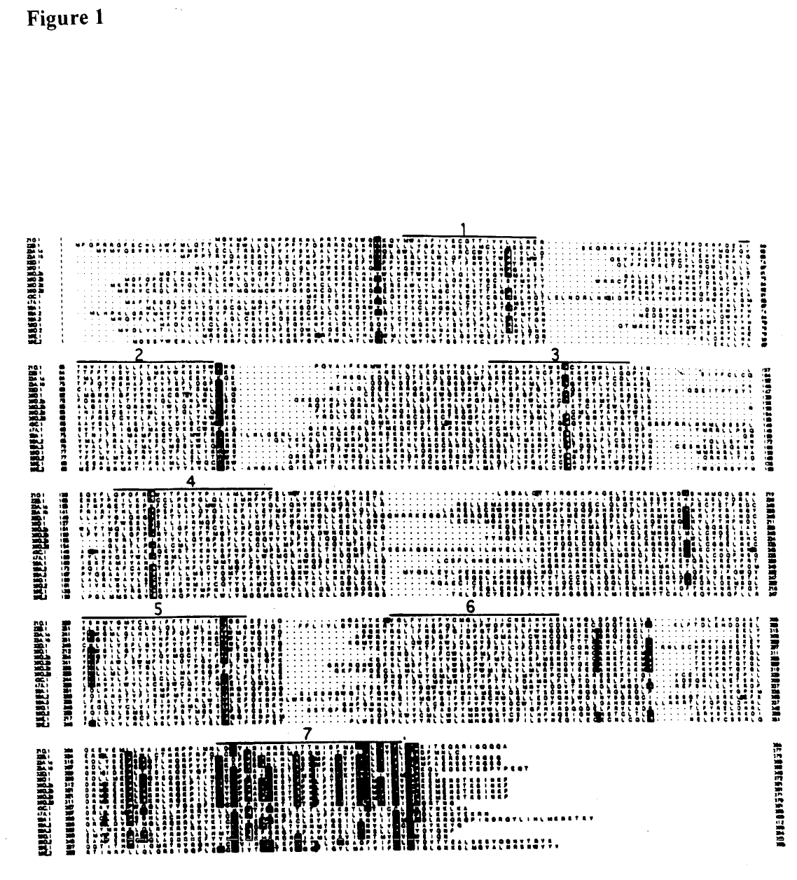

The proteins of the present invention have seven transmembrane domains as defined by hydropathy analysis (Kyte & Doolittle, (1982) J. Mol. Biol. 157, 105-132). Furthermore, the proteins of the present invention have conserved amino acid residues in defined domains of the protein. For example, the proteins of the present invention have at least one of the following conserved amino acids as depicted in FIG. 1, including but not limited to, Serine in the amino terminal domain; Phenylalanine in the first transmembrane domain; Arginine in the first extracellular loop; Leucine in the fourth transmembrane domain; Leucine in the third transmembrane domain; Glycine in the fifth transmembrane domain; Tyrosine in the fifth transmembrane domain; Leucine in the third extracellular loop; Phenylalanine in the third extracellular loop; Alanine in the seventh transmembrane domain; Glycine in the seventh transmembrane domain; Leucine in the seventh transmembrane domain; Aspartate in the seventh transmembrane domain; Alanine in the seventh transmembrane domain; Threonine in the seventh transmembrane domain; Tyrosine in the seventh transmembrane domain; Valine in the seventh transmembrane domain; Glutamine in the carboxy terminal domain; and Phenylalanine in the carboxy terminal domain. In addition, the conserved amino acids may be selected from any of the amino acid residues indicated as being conserved among Drosophila Gustatory Receptor (DGR) proteins as depicted in FIG. 1 (shaded).

Thus, the proteins of the present invention include molecules having the amino acid sequence disclosed in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92; fragments thereof having a consecutive sequence of at least about 3, 4, 5, 6, 10, 15, 20, 25, 30, 35 or more amino acid residues of the proteins, for instance, antigenic fragments such as those found in the extracellular loops of the protein (see FIG. 1); amino acid sequence variants wherein an amino acid residue has been inserted N- or C-terminal to, or within, the disclosed sequence; and amino acid sequence variants of the disclosed sequences, or their fragments as defined above, that have been substituted by another residue. Contemplated variants further include those containing predetermined mutations by, e.g., homologous recombination, site-directed or PCR mutagenesis, and the corresponding proteins of other insect species, including but not limited to the order Diptera, Lepidoptera, Homopterera and Coleoptera, within these orders, preferably the genus Drosophila, Anopheles, Aedes, Ceratitis, Muscidae, Culicidae, Anagasta and Popilla and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

As described below, members of the family of proteins can be used: 1) to identify agents which modulate at least one activity of the protein; 2) to identify binding partners for the protein, 3) as an antigen to raise polyclonal or monoclonal antibodies, and 4) in methods to modify insect behavior.

B. Nucleic Acid Molecules

The present invention further provides nucleic acid molecules which encode any of the proteins having SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92 and the related proteins herein described, preferably in isolated form. As used herein, “nucleic acid” is defined as RNA or DNA that encodes a protein or peptide as defined above, is complementary to a nucleic acid sequence encoding such peptides, hybridizes to such a nucleic acid and remains stably bound to it under appropriate stringency conditions, or encodes a polypeptide sharing at least 75% sequence identity, preferably at least 80%, and more preferably at least 85%, with the peptide sequences in conserved domains. Specifically contemplated are genomic DNA, cDNA, mRNA and antisense molecules, as well as nucleic acids based on alternative backbones or including alternative bases whether derived from natural sources or synthesized. Such hybridizing or complementary nucleic acids, however, are defined further as being novel and non-obvious over any prior art nucleic acid including that which encodes, hybridizes under appropriate stringency conditions, or is complementary to nucleic acid encoding a protein according to the present invention.

Homology or identity at the amino acid or nucleotide level is determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases (see Altschul et al., (1994) Nature Genetics 6, 119-129 which is fully incorporated by reference). The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively.

“Stringent conditions” are those that (1) employ low ionic strength and high temperature for washing, for example, 0.5 M sodium phosphate buffer at pH 7.2, 1 mM EDTA at pH 8.0 in 7% SDS at either 65° C. or 55° C., or (2) employ during hybridization a denaturing agent such as formamide, for example, 50% formamide with 0.1% bovine serum albumin, 0.1% Ficoll, 0.1% polyvinylpyrrolidone, 0.05 M sodium phosphate buffer at pH 6.5 with 0.75 M NaCl, 0.075 M sodium citrate at 42° C. Another example is use of 50% formamide, 5×SSC (0.75 M NaCl, 0.075 M sodium citrate), 50 mM sodium phosphate at pH 6.8, 0.1% sodium pyrophosphate, 5× Denhardt's solution, sonicated salmon sperm DNA (50 μg/ml), 0.1% SDS and 10% dextran sulfate at 55° C., with washes at 55° C. in 0.2×SSC and 0.1% SDS. A skilled artisan can readily determine and vary the stringency conditions appropriately to obtain a clear and detectable hybridization signal. Preferred molecules are those that hybridize under the above conditions to the complements of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91, and which encode a functional protein.

As used herein, a nucleic acid molecule is said to be “isolated” when the nucleic acid molecule is substantially separated from contaminant nucleic acid encoding other polypeptides from the source of nucleic acid. For information on how to extract and manipulate nucleic acids from Drosophila, see, for example, Roberts, (1998) Drosophila: A Practical Approach, IRL Press.

The present invention further provides fragments of any one of the encoding nucleic acids molecules. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments of the invention encode antigenic fragments such as the extracellular loops or N-terminal domain of the protein depicted in SEQ ID NO: 9 (21D.1) and as set forth in FIG. 1. If the fragment is to be used as a nucleic acid probe or PCR primer, then the fragment length is chosen so as to obtain a relatively small number of false positives during probing and priming.

Fragments of the encoding nucleic acid molecules of the present invention (i.e., synthetic oligonucleotides) that are used as probes or specific primers for the polymerase chain reaction (PCR), or to synthesize gene sequences encoding proteins of the invention can easily be synthesized by chemical techniques, for example, the phosphotriester method of Matteucci et al., (1981) J. Am. Chem. Soc. 103, 3185-3191) or using automated synthesis methods. In addition, larger DNA segments can readily be prepared by well known methods, such as synthesis of a group of oligonucleotides that define various modular segments of the gene, followed by ligation of oligonucleotides to build the complete modified gene.

The encoding nucleic acid molecules of the present invention may further be modified so as to contain a detectable label for diagnostic and probe purposes. A variety of such labels are known in the art and can readily be employed with the encoding molecules herein described. Suitable labels include, but are not limited to, fluorescent-labeled, biotin-labeled, radio-labeled nucleotides and the like. A skilled artisan can employ any of the art known labels to obtain a labeled encoding nucleic acid molecule.

Modifications to the primary structure itself by deletion, addition, or alteration of the amino acids incorporated into the protein sequence during translation can be made without destroying the activity of the protein. Such substitutions or other alterations result in proteins having an amino acid sequence encoded by a nucleic acid falling within the contemplated scope of the present invention.

C. Isolation of Other Related Nucleic Acid Molecules

As described above, the identification and characterization of the nucleic acid molecules having SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91 allows a skilled artisan to isolate nucleic acid molecules that encode other members of the protein family in addition to the sequences herein described. Further, the presently disclosed nucleic acid molecules allow a skilled artisan to isolate nucleic acid molecules that encode other members of the family of proteins in addition to the protein having SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92.

Essentially, a skilled artisan can readily use any one of the amino acid sequences selected from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92, to generate antibody probes to screen expression libraries prepared from appropriate cells. Typically, polyclonal antiserum from mammals such as rabbits immunized with the purified protein (as described below) or monoclonal antibodies can be used to probe a cDNA or genomic expression library to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any organism. Oligomers containing approximately 18-20 nucleotides (encoding about a six to seven amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules. For example, degenerate primers can be used to clone any Drosophila Gustatory Receptor (DGR) gene across species. Specifically, based on the sequence information derived from the family of Drosophila Gustatory Receptors, degenerate primers can be designed based on conserved sequences among gustatory receptors, which can then be used to clone nucleic acid molecules encoding gustatory receptor proteins from other species of insects.

Applicants have also identified a method for isolating nucleic acid molecules that encode other members of the protein family in addition to the sequences herein described. Essentially, a two-step strategy is employed to identify gustatory receptor genes from the genomic database. First, a computer algorithm was designed to search genomic sequences for open reading frames (ORFs) from candidate gustatory receptor genes. Second, RT-PCR is used to determine if transcripts from any of these ORFs are expressed in gustatory organs.

The algorithm is used to identify G protein-coupled receptors (GPCR) genes using statistical characterization of amino acid physico-chemical profiles in combination with a non-parametric discriminant function. The algorithm is trained on a set of putative sequences from a database. In the first step, three sets of descriptors are used to summarize the physico-chemical profiles of the sequences. These are GES scale of hydropathy (Engelman et al., (1986) Annu. Rev. Biophys. Biophys. Chem. 15, 321-353), polarity (Brown, (1991) Molecular Biology Labfax, Academic Press), and amino acid usage frequency. For the first two of these measurements, a computed sliding window profile is employed (White, (1994) Membrane Protein Structure, Oxford University Press) using a kernel of a certain number of amino acids as a constant function convoluted with a certain number of amino acids as a Gaussian function. These profiles are then summarized with three statistics; the periodicity, average derivative and the variance of the derivative.

Each sequence is then characterized by multiple variables using a non-parametric linear discriminant function that is optimized to separate the known family proteins from random proteins in the training set. The same linear discriminant function with the scores derived from the training set is used to screen any nucleic acid database for candidate genes. The candidate sequences are given significance values by an odds ratio of the proteins and non-family proteins, computed using the observed empirical distribution of the training set. Those sequences with a sufficiently high odds ratio are considered for further analysis. The algorithm can also be used to identify any protein family by altering the training set of sequences.

The method of identification further includes steps for identifying novel gustatory receptor genes comprising selecting candidate gustatory receptor genes by screening a nucleic acid database using an algorithm trained to identify seven transmembrane receptors genes; screening said selected candidate gustatory receptor genes by identifying nucleic acid sequences with conserved amino acid residues and intron-exon boundaries common to gustatory receptors, and open reading frames of sufficient size as to encode a seven transmembrane receptor. As an additional step, the expression of gustatory receptor genes is measured to confirm candidate gustatory gene as an gustatory gene. The exon-intron boundaries and conserved amino acid residues may be selected from any of the positions depicted in FIG. 3. Alternatively, selecting candidate gustatory receptor genes by screening a nucleic acid database for nucleic acid sequences with sufficient homology to at least one known gustatory receptor gene is also encompassed in the invention. In a preferred embodiment, the nucleic acid database is a genomic database, an EST database or even an gustatory receptor database as previously described (Skoufos et al., (1999) Nucleic Acids Research 27, 343-345).

In one example of the invention, the training set could consist of known gustatory receptors from Drosophila and could be used to search genomic sequences for new gustatory receptors in other species. In a similar example, the training set could consist of known sequences coding for receptors from a particular family and could be used to identify homologs across species. Specifically, gustatory receptors of one species could be used as a training set to identify gustatory receptors in another species.

D. rDNA Molecules Containing a DNA Molecule

The present invention further provides recombinant DNA molecules (rDNAs) that contain a coding sequence. As used herein, a rDNA molecule is a DNA molecule that has been subjected to molecular manipulation in vitro. Methods for generating rDNA molecules are well known in the art, for example, see Sambrook et al., (1989) Molecular Cloning—A Laboratory Manual, Cold Spring Harbor Laboratory Press. In the preferred rDNA molecules, a coding DNA sequence is operably linked to expression control sequences or vector sequences.

The choice of vector and expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from BioRad Laboratories, pPL and pKK223 available from Pharmacia.

Expression vectors compatible with eukaryotic cells, preferably those compatible with invertebrate cells such as insect cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors. Vectors may be modified to include insect cell specific promoters if needed.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene (Southern et al., (1982) J. Mol. Appl. Genet. 1, 327-341). Alternatively, the selectable marker can be present on a separate plasmid, and the two vectors are introduced by co-transfection of the host cell, and selected by culturing in the appropriate drug for the selectable marker.

E. Host Cells Containing an Exogenously Supplied Coding Nucleic Acid

The present invention further provides host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably insect cells such as those from a Drosophila cell line. Preferred Drosophila host cells include Drosophila Schneider line 2, and the like insect tissue culture cell lines. Any prokaryotic host can be used to express a rDNA molecule encoding a protein of the invention. The preferred prokaryotic host is E. coli.

Transformation of appropriate cell hosts with a rDNA molecule of the present invention is accomplished by well known methods that typically depend on the type of vector used and host system employed. With regard to transformation of prokaryotic host cells, electroporation and salt treatment methods are typically employed, see, for example, Cohen et al., (1972) Proc. Natl. Acad. Sci. USA 69, 2110-2114; and Sambrook et al., (1989) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al., (973) Virology 52, 456-467; Wigler et al., (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, (1975) J. Mol. Biol. 98, 503-517; or Berent et al., (1985) Biotech. Histochem. 3, 208; or the proteins produced from the cell assayed via an immunological method.

F. Production of Recombinant Proteins Using a rDNA Molecule

The present invention further provides methods for producing a protein of the invention using nucleic acid molecules herein described. In general terms, the production of a recombinant form of a protein typically involves the following steps: First, a nucleic acid molecule is obtained that encodes a protein of the invention, such as any of the nucleic acid molecule depicted in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host-expression system known in the art for use with the nucleic acid molecules of the invention to produce recombinant protein.

G. Methods to Identify Binding Partners

Another embodiment of the present invention provides methods for use in isolating and identifying binding partners of any of the DGR proteins of the invention. In detail, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analyzed. To identify and isolate a binding partner, the entire protein, for instance a protein comprising the entire amino acid sequence of any of the proteins depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92 can be used. Alternatively, a fragment of any of the proteins can be used.

As used herein, a cellular extract refers to a preparation or fraction which is made from a lysed or disrupted cell. The preferred source of cellular extracts will be cells derived from Drosophila, for instance, labellar cellular extract.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with any of the proteins of the invention under conditions in which association of the protein with the binding partner can occur. A variety of conditions can be used, the most preferred being conditions that closely resemble conditions found in the cytoplasm of a Drosophila cell. Features such as osmolarity, pH, temperature and the concentration of cellular extract used, can be varied to optimize the association of the protein with the binding partner.

As used herein, the term “binding partner” refers to any molecule that binds to a DGR protein of the invention. Binding partners to any one of the Gustatory receptors of the invention include, but are not limited to, small molecules, peptides, polypeptides and proteins. In one embodiment, the binding partner is a co-receptor that forms a dimer complex with the Gustatory receptor, such complexes being necessary for efficient signal transduction. In another embodiment, the binding partner can be a G protein or a subunit of a G protein as the Gustatory receptors of the invention are assumed to be G protein-linked because of their seven transmembrane domains.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density-sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide-binding partner pairs from other constituents found in the extract. The identified binding partners can be either a single protein or a complex made up of two or more proteins. Alternatively, binding partners may be identified using a Far-Western assay according to the procedures of Takayama et al., (1997) Methods Mol. Biol. 69, 171-184 or identified through the use of epitope tagged proteins or GST fusion proteins.

Alternatively, the nucleic acid molecules of the invention can be used in a yeast two-hybrid system. The yeast two-hybrid system has been used to identify other protein partner pairs (Alifragis et al., (1997) Proc. Natl. Acad. Sci. USA 94, 13099-13104; Dong et al., (1999) Gene 237, 421-428) and can readily be adapted to employ the nucleic acid molecules herein described.

In another embodiment, binding partners may be identified in insects using single unit recordings as previously described (Kaissling, (1995) Single unit recordings in insect gustatory organs, in: Spielman & Brand, (1995) Experimental Cell Biology of Taste and Olfaction, CRC Press). Using single unit recordings in vivo, response profiles are established for potential ligands, these profiles are then categorized into distinct functional classes indicative of distinct receptor-ligand interactions (see, e.g., U.S. Pat. No. 5,993,778). Single unit recordings in transgenic insects which contain transgenes resulting in over- or under-expression of a gene are also useful for identifying and characterizing ligands which bind to multiple gustatory receptors as well as identifying and characterizing new gustatory receptors.

The nucleic acids of the invention and their corresponding proteins can be used on an array or microarray for high-throughput screening for agents which interact with either the nucleic acids of the invention or their corresponding proteins.

An “array” or “microarray” generally refers to a grid system which has each position or probe cell occupied by a defined nucleic acid fragments also known as oligonucleotides. The arrays themselves are sometimes referred to as “chips” or “biochips”. High-density nucleic acid and protein microarrays often have thousands of probe cells in a variety of grid styles. For DNA microarray protocols particularly suited to studying Drosophila, see, for example, Sullivan et al., (2000) Drosophila Protocols, Cold Spring Harbor Laboratory Press.

A typical molecular detection chip includes a substrate on which an array of recognition sites, binding sites or hybridization sites are arranged. Each site has a respective molecular receptor which binds or hybridizes with a molecule having a predetermined structure. The solid support substrates which can be used to form surface of the array or chip include organic and inorganic substrates, such as glass, polystyrenes, polyimides, silicon dioxide and silicon nitride. For direct attachment of probes to the electrodes, the electrode surface must be fabricated with materials capable of forming conjugates with the probes.

Once the array is fabricated, a sample solution is applied to the molecular detection chip and molecules in the sample bind or hybridize at one or more sites. The sites at which binding occurs are detected, and one or more molecular structures within the sample are subsequently deduced. Detection of labeled batches is a traditional detection strategy and includes radioisotope, fluorescent and biotin labels, but other options are available, including electronic signal transduction.

Polymer arrays of nucleic acid probes can be used to extract information from, for example, nucleic acid samples. These samples are exposed to the probes under conditions that permit binding. The arrays are then scanned to determine to which probes the sample molecules have interacted with the nucleic acids of the polymer array. One can obtain information by careful probe selection and using algorithms to compare patterns of interactions. For example, the method is useful in screening for novel gustatory receptors in multiple organisms. For example, Drosophila degenerate gustatory receptor oligonucleotide arrays can be used to examine a nucleic acid sample from another insect species in order to identify novel gustatory receptors in that species.

In typical applications, a complex solution containing one or more substances to be characterized contacts a polymer array comprising nucleic acids. For example, the array is comprised of nucleic acid probes. The probes of the array can be either DNA or RNA, which may be either single-stranded or double-stranded. In a preferred embodiment of the invention, the probes are arranged (either by immobilization, typically by covalent attachment, of a pre-synthesized probe or by synthesis of the probe on the substrate) on the substrate or chips in lanes stretching across the chip and separated, and these lanes are in turned arranged in blocks of preferably five lanes, although blocks of other sizes will have useful application. The present invention provides individual probes, sets of probes, and arrays of probe sets on chips, in specific patterns which are used to characterize the substances in a complex mixture by producing a distinct image which is representative of the binding interactions between the probes on the chip and the substances in the complex mixture. The pattern of hybridization to the chip allows inferences to be drawn about the substances present in the complex mixture.

The substances in the complex solution will bind to the nucleic acids on the array. The substances of the complex mixture which bind to the nucleic acids of the array may include, but are not limited to, complementary nucleic acids, non-complementary nucleic acids, proteins, antibodies, oligosaccharides, etc. The types of binding may include, but are not limited to, specific and non-specific, competitive and non-competitive, allosteric, cooperative, non-cooperative, complementary and non-complementary, etc. For example, the nucleic acids of the array can bind to complementary nucleic acids in the complex mixture but can also bind in a tertiary manner, independent of base pairing, to non-complementary nucleic acids.

The nucleic acids of the array or the substances of the complex mixture may be tagged with a detectable label. The detectable label can be, for example, a luminescent label, a light scattering label or a radioactive label. Accordingly, locations at which substances interact can be identified by either determining if the signal of the label has been quenched by binding or identifying locations where the signal of the label is present in cases where the substances of the complex mixture have been labeled. Based on the locations where binding is detected, information regarding the complex mixture can be obtained.

The methods of this invention will find particular use wherever high through-put of samples is required. In particular, this invention is useful in ligand screening settings and for determining the composition of complex mixtures.

Polypeptides are an exemplary system for exploring the relationship between structure and function in biology. When the twenty naturally occurring amino acids are condensed into a polymeric molecule they form a wide variety of three-dimensional configurations, each resulting from a particular amino acid sequence and solvent condition. For example, the number of possible polypeptide configurations using the twenty naturally occurring amino acids for a polymer five amino acids long is over three million. Typical proteins are more than one-hundred amino acids in length.

In typical applications, a complex solution containing one or more substances to be characterized contacts a polymer array comprising polypeptides. The polypeptides of the invention can be prepared by classical methods known in the art, for example, by using standard solid phase techniques. The standard methods include exclusive solid phase synthesis, partial solid phase synthesis methods, fragment condensation, classical solution synthesis and recombinant DNA technology (see Merrifield, (1963) Am. Chem. Soc. 85, 2149-2152). On solid phase, the synthesis is typically commenced from the C-terminal end of the peptide using an alpha-amino protected resin. A suitable starting material can be prepared, for instance, by attaching the required alpha-amino acid to a chloromethylated resin, a hydroxy-methyl resin or a benzhydrylamine resin.

The alpha-amino protecting groups are those known to be useful in the art of stepwise synthesis of peptides. Included are acyl type protecting groups, aromatic urethane type protecting groups, aliphatic urethane protecting groups and alkyl type protecting groups. The side chain protecting group remains intact during coupling and is not split off during the deprotection of the amino-terminus protecting group or during coupling. The side chain protecting group must be removable upon the completion of the synthesis of the final peptide and under reaction conditions that will not alter the target peptide.

After removal of the alpha-amino protecting group, the remaining protected amino acids are coupled stepwise in the desired order. An excess of each protected amino acid is generally used with an appropriate carboxyl group activator such as dicyclohexylcarbodiimide (IDCC) in solution, for example, in methylene chloride, dimethyl formamide (DMF) mixtures.

In a preferred embodiment, the polypeptides or proteins of the array can bind to other co-receptors to form a heteroduplex on the array. In yet another embodiment, the polypeptides or proteins of the array can bind to peptides or small molecules.

These procedures can also be used to synthesize peptides in which amino acids other than the twenty naturally occurring, genetically encoded amino acids are substituted at one, two, or more positions of any of the compounds of the invention. For instance, naphthylalanine can be substituted for tryptophan, facilitating synthesis. Other synthetic amino acids that can be substituted into the peptides of the present invention include L-hydroxypropyl, L-3, 4-dihydroxyphenylalanyl, d-amino acids such as L-d-hydroxylysyl and D-d-methylalanyl, L-α-methylalanyl and β-amino acids non-naturally occurring synthetic amino acids can also be incorporated into the peptides of the present invention (see Roberts et al., (1983) Peptide Synthesis 5, 341-449).

One can replace the naturally occurring side chains of the twenty genetically encoded amino acids (or D amino acids) with other side chains, for instance with groups such as alkyl, lower alkyl, cyclic four, five, six, to seven-membered alkyl, amide, amide lower alkyl, amide di(lower alkyl), lower alkoxy, hydroxy, carboxy and the lower ester derivatives thereof, and with four, five, six, to seven-membered heterocyclic. In particular, proline analogs in which the ring size of the proline residue is changed from five members to four, six or seven members can be employed. Cyclic groups can be saturated or unsaturated, and if unsaturated, can be aromatic or non-aromatic. Heterocyclic groups preferably contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl, oxazolyl, piperazinyl, piperidyl, pyranyl, pyrazinyl, pyrazolidinyl, pyrazolinyl, pyrazolyl, pyridazinyl, pyridyl, pyrimidinyl, pyrrolidinyl, pyrrolinyl, pyrrolyl, thiadiazolyl, thiazolyl, thienyl, thiomorpholinyl and triazolyl. These heterocyclic groups can be substituted or unsubstituted. Where a group is substituted, the substituent can be alkyl, alkoxy, halogen, oxygen, or substituted or unsubstituted phenyl.

One can also readily modify the peptides of the instant invention by phosphorylation (see Bannwarth et al., (1996) Biorg. Med. Chem. Let. 6, 2141-2146) and other methods for making peptide derivatives of the compounds of the present invention are described in Hruby et al., (1990) Biochem. J. 268, 249-262). Thus, the peptide compounds of the invention also serve as a basis to prepare peptide mimetics with similar biological activity. The array can also comprise peptide mimetics with the same or similar desired biological activity as the corresponding peptide compound but with more favorable activity than the peptide with respect to solubility, stability, and susceptibility to hydrolysis and proteolysis (see Morgan et al., (1989) Ann. Rep. Med. Chem. 24, 243-252).

Peptides suitable for use in this embodiment generally include those peptides, for example, ligands, that bind to a receptor, such as seven transmembrane proteins. Such peptides typically comprise about 150 amino acid residues or less and, more preferably, about 100 amino acid residues or less. Polypeptides or proteins suitable for use in this embodiment generally include those polypeptides or proteins that interact with a receptor, such as a co-receptor or G protein. Such polypeptides or proteins typically comprise about 150 amino acid residues or more and, more preferably, about 400 amino acids or more.

The peptides of the present invention may exist in a cyclized form with an intramolecular disulfide bond between the thiol groups of the cysteines. Alternatively, an intermolecular disulfide bond between the thiol groups of the cysteines can be produced to yield a dimeric (or higher oligomeric) compound. One or more of the cysteine residues may also be substituted with a homocysteine. Other embodiments of this invention provide for analogs of these disulfide derivatives in which one of the sulfurs has been replaced by a CH₂ group or other isostere for sulfur. These analogs can be made via an intramolecular or intermolecular displacement, using methods known in the art.

H. Methods to Identify Agents that Modulate Expression of DGRs.

Another embodiment of the present invention provides methods for identifying agents that modulate the expression of a nucleic acid encoding any one of the DGRproteins of the invention such as any protein having the amino acid sequence depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92. Such assays may utilize any available means of monitoring for changes in the expression level of the nucleic acids of the invention. As used herein, an agent is said to modulate the expression of a nucleic acid of the invention, for instance a nucleic acid encoding any one of the proteins having the amino acid sequence depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92, if it is capable of up- or down-regulating expression of the nucleic acid in a cell.

In one assay format, cell lines that contain reporter gene fusions between the open reading frame of any one of the nucleotides depicted in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91, and any assay fusion partner may be prepared. Numerous assay fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding at least one of the proteins having the sequence depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92.

Additional assay formats may be used to monitor the ability of the agent to modulate the expression of a nucleic acid encoding at least one protein of the invention selected from the group of proteins having SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press.

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementary nucleotides which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., (1989) Molecular Cloning—A Laboratory Manual, Cold Spring Harbor Laboratory Press; or Ausubel et al., (1995) Current Protocols in Molecular Biology, Greene Publishing Company.

Hybridization conditions are modified using known methods, such as those described by Sambrook et al., (1989) and Ausubel et al., (1995) as required for each probe. Hybridization of total cellular RNA or RNA enriched for polyA+ RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass wafer can then be exposed to total cellular RNA or polyA RNA from a sample under conditions in which the affixed sequences will specifically hybridize. Such glass wafers and hybridization methods are widely available, for example, those disclosed by Beattie, 1995 (WO 9511755). By examining for the ability of a given probe to specifically hybridize to an RNA sample from an untreated cell population and from a cell population exposed to the agent, agents which up- or down-regulate the expression of a nucleic acid encoding at least one protein having the amino acid sequence depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92 are identified.

Hybridization for qualitative and quantitative analysis of mRNA may also be carried out by using a RNase Protection Assay (i.e., RPA, see Ma et al., (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 μg/ml ribonuclease A and 2 μg/ml ribonuclease. After deactivation and extraction of extraneous proteins, the samples are loaded onto urea-polyacrylamide gels for analysis.

In another assay format, agents which effect the expression of the instant gene products, cells or cell lines would first be identified which express said gene products physiologically. Cells and cell lines so identified would be expected to comprise the necessary cellular machinery such that the fidelity of modulation of the transcriptional apparatus is maintained with regard to exogenous contact of agent with appropriate surface transduction mechanisms and the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag. Such a process is well known in the art (see Sambrook et al., (1989) Molecular Cloning—A Laboratory Manual, Cold Spring Harbor Laboratory Press).

Cells or cell lines transduced or transfected as outlined above would then be contacted with agents under appropriate conditions; for example, the agent comprises an acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells will be disrupted and the polypeptides from disrupted cells are fractionated such that a polypeptide fraction is pooled and contacted with an antibody to be further processed by immunological assay (e.g., ELISA, immunoprecipitation or Western blot). The pool of proteins isolated from the “agent contacted” sample will be compared with a control sample where only the excipient is contacted with the cells and an increase or decrease in the immunologically generated signal from the “agent contacted” sample compared to the control will be used to distinguish the effectiveness of the agent.

I. Methods to Identify Agents that Modulate Activity of DGRs

Another embodiment of the present invention provides methods for identifying agents that modulate at least one activity of a protein of the invention such as any one of the proteins having the amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92. Such methods or assays may utilize any means of monitoring or detecting the desired activity including, but not limited to, behavioral and electrophysiological studies.

In one format, the relative amounts of a protein of the invention are expressed in a cell population that has been exposed to the agent to be tested and is compared to an un-exposed control cell population. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or if desired, required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co., may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for some applications, use of monoclonal preparations is preferred. Immortalized cell lines which secrete the desired monoclonal antibodies may be prepared using the standard method of Kohler & Milstein, (1975) Nature 256, 495-497 or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal or polyclonal antisera which contain the immunologically significant portion can be used as antagonists, as well as the intact antibodies. Use of immunologically reactive fragments, such as the Fab, Fab′ of F(ab′)₂ fragments is often preferable, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, particularly humanized antibodies.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a non-random basis which takes into account the sequence of the target site and its conformation in connection with the agent's action. Agents can be rationally selected or rationally designed by utilizing the peptide sequences to identify proposed binding motifs, glycosylation and phosphorylation sites on the protein.

The agents of the present invention can be, as examples, peptides, small molecules, vitamin derivatives, as well as carbohydrates. A skilled artisan can readily recognize that there is no limit as to the structural nature of the agents of the present invention. Dominant-negative proteins, DNA encoding these proteins, antibodies to these proteins, peptide fragments of these proteins or mimics of these proteins may be contacted with cells to affect function. “Mimic” as used herein refers to the modification of a region or several regions of a peptide molecule to provide a structure chemically different from the parent peptide but topographically and functionally similar to the parent peptide (see Meyers, (1995) Molecular Biology & Biotechnology, VCH Publishers).

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies.

J. Transgenic Organisms

Transgenic insects containing mutant, knock-out or modified genes corresponding to any one of the cDNA sequences depicted in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91 are also included in the invention. Transgenic insects are genetically modified insects into which recombinant, exogenous or cloned genetic material has been experimentally transferred. Such genetic material is often referred to as a “transgene”. The nucleic acid sequence of the transgene, in this case a form of any one of the sequences depicted in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91, may be integrated either at a locus of a genome where that particular nucleic acid sequence is not otherwise normally found or at the normal locus for the transgene. The transgene may consist of nucleic acid sequences derived from the genome of the same species or of a different species than the species of the target insect.

The term “germ cell line transgenic insect” refers to a transgenic insect in which the genetic alteration or genetic information was introduced into a germ line cell, thereby conferring the ability of the transgenic insect to transfer the genetic information to offspring. If such offspring in fact possess some or all of that alteration or genetic information, then they too are transgenic insects.

The alteration or genetic information may be foreign to the species of insect to which the recipient belongs, foreign only to the particular individual recipient, or may be genetic information already possessed by the recipient. In the last case, the altered or introduced gene may be expressed (i.e., over-expression and knock-out) differently than the native gene.

Transgenic insects can be produced by a variety of different methods including P element-mediated transformation by microinjection (see, e.g., Rubin & Spradling, (1982) Science 218, 348-353; Orr & Sohal, (1993) Arch. Biochem. Biophys. 301, 34-40), transformation by microinjection followed by transgene mobilization (Mockett et al., (1999) Arch. Biochem. Biophys. 371, 260-269), electroporation (Huynh & Zieler, (1999) J. Mol. Biol. 288, 13-20) and through the use of baculovirus (Yamao et al., (1999) Genes Dev. 13, 511-516. Furthermore, the use of adenoviral vectors to direct expression of a foreign gene to gustatory neuronal cells can also be used to generate transgenic insects (see, e.g., Holtmaat et al., (1996) Brain. Res. Mol. Brain Res. 41, 148-156).

A number of recombinant or transgenic insects have been produced, including those which over-express superoxide dismutase (Mockett et al., (1999) Arch. Biochem. Biophys. 371, 260-269); express Syrian hamster prion protein (Raeber et al., (1995) Mech. Dev. 51, 317-327); express cell-cycle inhibitory peptide aptamers (Kolonin & Finley (1998) Proc. Natl. Acad. Sci. USA 95, 14266-14271); and those which lack expression of the putative ribosomal protein S3A gene (Reynaud et al., (1997) Mol. Gen. Genet. 256, 462-467).

While insects remain the preferred choice for most transgenic experimentation, in some instances it is preferable or even necessary to use alternative animal species. Transgenic procedures have been successfully utilized in a variety of animals, including mice, rats, sheep, goats, pigs, dogs, cats, monkeys, chimpanzees, hamsters, rabbits, cows and guinea pigs (see, e.g., Kim et al., (1997) Mol. Reprod. Dev. 46, 515-526; Houdebine, (1995) Reprod. Nutr. Dev. 35, 609-617; Petters, (1994) Reprod. Fertil. Dev. 6, 643-645; Schnieke et al., (1997) Science 278, 2130-2133; and Amoah, (1997) J. Anim. Sci. 75, 578-585).

The method of introduction of nucleic acid fragments into insect cells can be by any method which favors co-transformation of multiple nucleic acid molecules. For instance, Drosophila embryonic Schneider line 2 (S2) cells can be stably transfected as previously described (Schneider, (1972) J. Embryol. Exp. Morphol. 27, 353-365). Detailed procedures for producing transgenic insects are readily available to one skilled in the art (see Rubin & Spradling, (1982) Science 218, 348-353; Orr & Sohal, (1993) Arch. Biochem. Biophys. 301, 34-40, herein incorporated by reference in their entirety).

K. Uses for Agents that Modulate at Least One Activity of DGRs

1. Introduction.

Organisms, including insects, are continually exposed to a great number of gustatory stimuli released by other organisms as well as by other aspects of their environment. The gustatory receptor genes of the present invention play an important role in the detection and processing of these chemical stimuli, some of which have been implicated in initiating and modulating host-seeking and other behaviors, such as mating behaviors (see, for example, Roth, (1951) Ann. Entomol. Soc. Am. 44, 59-74; Jones et al., (1976) Ent. Exp. Appn. 19, 19-22; Gillies, (1980) Bull. Ent. Res. 70, 525-532; Kline et al., (1991) J. Med. Entomol. 28, 254-258).

Most importantly, the DGR genes of the present invention may be used to track down gustatory receptor genes in insects that damage crops or transmit diseases. The present invention provides the tools and methodologies for finding specific compounds that interfere with the insects' ability to detect tastes.

Of course, the present invention has important implications for improved methods of using pheromones and other semiochemicals for pest control. In addition, recent advancements in many other fields have greatly increased the variety of additional technologies for which the present invention also has significant applications. Examples of such advancements include, but are not limited to the following: (a) the development and application of new techniques of chemical identification and synthesis; (b) new chemical release techniques; (c) more sophisticated application technologies; and (d) more detailed information about the behavior of specific organisms.

While not wishing to be bound by the specific embodiments discussed herein, the following sections provide an overview of the wide variety of applications for which the present invention may be employed.

2. Definitions.

As used herein, the term “allomones” refers to any chemical substance produced or acquired by an organism that, when it contacts an individual of another species, evokes in the receiver a behavioral or developmental reaction adaptively favorable to the transmitter.

As used herein, the term “host” refers to any organism on which another organism depends for some life function. Examples of hosts include, but are not limited to, humans which may serve as a host for the feeding of certain species of mosquito and the leaves of soybeans (Glycine max (L.)) which may act as hosts for the oviposit of the green cloverworm (Plathypena scabra (F.)).

As used herein, the term “kairomones” refers to any of a heterogeneous group of chemical messengers that are emitted by organisms of one species but benefit members of another species. Examples include, but are not limited to, attractants, phagostimulants, and other substances that mediate the positive responses of, for example, predators to their prey, herbivores to their food plants, and parasites to their hosts. Kairomones suitable for the purposes of the invention and methods of obtaining them are described, for example, Hedin, (1985) Bioregulators for Pest Control, American Chemical Society.

As used herein, the term “pheromone” refers to a substance, or characteristic mixture of substances, that is secreted and released by an organism and detected by a second organism of the same or a closely related species, in which it causes a specific reaction, such as a definite behavioral reaction or a developmental process. Examples include, but are not limited to, the mating pheromones of fungi and insects. More than a thousand moth sex pheromones (Toth et al., (1992) J. Chem. Ecol. 18, 13-25; Arn et al., (1998) Appl. Entomol. Zoo. 33, 507-511) and hundreds of other pheromones have now been identified, including aggregation pheromones from beetles and other groups of insects. Various compositions, including resins and composite polymer dispensers, have been developed for the controlled release of pheromones have been developed (see, e.g., U.S. Pat. Nos. 5,750,129 & 5,504,142).

As used herein, the term “semiochemical” refers to any chemical substance that delivers a message or signal from one organism to another. Examples of such chemicals include, but are not limited to, pheromones, kairomones, oviposition deterrents, or stimulants, and a wide range of other classes of chemicals (see, for example, Nordlund et al., (1981) Semiochemicals: Their Role in Pest Control, John Wiley; Howse et al., (1998) Insect Pheromones and Their Use in Pest Management, Chapman & Hall).

As used herein, the term “synomones” refers to any chemical substance which benefits both the emitter and receiver. Examples include, but are not limited to, compounds involved in floral attraction of pollinators and species-isolating mechanisms, such as sex pheromones of related species, where an inhibitor often functions to prevent mating among sympatric species.

As used herein, the term “volatile” refers to a chemical which evaporates readily at those temperatures and pressures which are considered the relevant temperatures and pressures for the reference organism of interest.

3. As Tools for Further Scientific Research.

Identification of Gustatory Receptor Genes in Other Organisms. The algorithms of the present invention may be used directly to search for gustatory receptor genes in other organisms, as explained elsewhere herein.

Alternatively, nucleic acid probes or primers may be designed based on the DGR genes of the present invention. Such probes or primers may be used to identify and isolate gustatory receptor genes in other organisms. Methods of creating and using the necessary nucleic acid probes and primers are discussed elsewhere herein.

The highest probability of success in locating gustatory genes in other organisms using the DGR genes of the present invention will most likely occur by using a boot-strapping or leap-frogging method. Such methods involve first probing organisms most related to fruit flies and successively progressing to more unrelated organisms, using the most newly identified gustatory receptor genes to identify similar genes in the next, more unrelated, insect of interest. Thus, the first organisms to probe with the DGR genes of the present invention most preferably may be other flies from the order Diptera (i.e., the two-winged or true flies). Examples of suitable flies include, but are not limited to, the tsetse fly, horse fly, house fly, bluebottle fly, hover fly and mosquito. Dipterans which transmit diseases causing serious health problems are of particular interest (e.g., horse fly, tsetse fly, mosquito).

After the identification of gustatory receptor genes in various Diptera insects, the next organisms to probe most preferably may be from orders within the same subclass as Diptera. Finally, the next insects to use would be those from orders not within the same subclass as Diptera.

The insects which cause substantial health risks, crop damage, or other significant damage (e.g., to housing structure or cotton clothing) may be the most desirable targets for such studies. Examples of such insects include, but are not limited to, green cloverworm, Mexican bean beetle, potato leafhopper, corn earworm, green stink bug, northern corn rootworm, western corn rootworm, cutworms, wireworms, thrips, fleas, aphids (e.g., pea aphid, spotted alfalfa aphid), European corn borer, fall armyworm, southwestern corn borer, grasshoppers, Japanese beetle, termites, leafhoppers (e.g., potato leafhopper, three-cornered alfalfa hopper), stink bugs, crickets, Hessian fly, greenbugs and weevils (e.g., alfalfa weevil, bollweevil).

Gustatory receptor genes identified by this process may then be used to screen non-Insecta organisms for gustatory receptor genes. Organisms of interest may include, but are not limited to, mites, ticks, spiders, nematodes, centipedes, mice, rats, salmon, pigeons, dogs, horses and humans. In a preferred embodiment, the gustatory receptor genes identified by this process would be used to identify gustatory receptor genes in humans.

Genetic Manipulations. The tools and methodologies of the present invention may be used by neurobiologists to probe more complex workings of an organism's response system, including those of a mammal's brain.

Knock-outs. By systematically knocking out and analyzing the expression patterns of the gustatory receptor genes of the present invention and observing the effects on taste sensitivity and behavior, researchers will be able to piece together a wiring diagram of the gustatory system of the fruit fly.

The term “knock-out” generally refers to mutant organisms which contain a null allele of a specific gene. Methods of making knock-out or disruption transgenic animals, especially mice, are generally known by those skilled in the art and are discussed herein and elsewhere (see, for example, the section herein entitled Transgenic Organisms and the following: Manipulating the Mouse Embryo, (1986) Cold Spring Harbor Laboratory Press; Capecchi, (1989) Science 244, 1288-1292; Li et al., (1995) Cell 80, 401-411; U.S. Pat. Nos. 5,981,830 & 5,789,654, each of which is incorporated herein by reference.

Parallel studies may be conducted in other organisms by using the gustatory receptor genes and the methods of the present invention to identify the gustatory receptor genes of other organisms and then creating knock-outs for the gustatory receptor genes of those organisms.

Disabling Genes. Using the gustatory receptor genes of the present invention, it is now possible to selectively disable specific DGR genes and look for changes in taste response and behavior. Parallel studies may be conducted in other organisms by using the gustatory receptor genes and the methods of the present invention to identify the gustatory receptor genes of other organisms and then disabling gustatory receptor genes of those organisms.

Methods of disabling genes are generally known by those skilled in the art. An example of an effective disabling modification would be a single nucleotide deletion occurring at the beginning of a gustatory receptor gene that would produce a translational reading frameshift. Such a frameshift would disable the gene, resulting in non-expressible gene product and thereby disrupting functional protein production by that gene.

In addition to disabling genes by deleting nucleotides, causing a transitional reading frameshift, disabling modifications would also be possible by other techniques including insertions, substitutions, inversions or transversions of nucleotides within the gene's DNA that would effectively prevent the formation of the protein coded for by the DNA.

It is also within the capabilities of one skilled in the art to disable genes by the use of less specific methods. Examples of less specific methods would be the use of chemical mutagens such as hydroxylamine or nitrosoguanidine or the use of radiation mutagens such as gamma radiation or ultraviolet radiation to randomly mutate genes, such as the DGR genes of the present invention. Such mutated strains could, by chance, contain disabled gustatory receptor genes such that the genes are no longer capable of producing functional proteins for any one or more of the domains. The presence of the desired disabled genes could be detected by routine screening techniques. For further guidance, see U.S. Pat. No. 5,759,538.

Over-expression. Using the gustatory receptor genes of the present invention, it is now possible to selectively over-express specific DGR genes and look for changes in taste response and behavior. Parallel studies may be conducted in other organisms by using the gustatory receptor genes and the methods of the present invention to identify the gustatory receptor genes of other organisms and then overexpress the gustatory receptor genes of those organisms.

Methods of overexpressing genes are generally known by those skilled in the art. For examples of producing cells which overexpress specific genes, see, for example, U.S. Pat. Nos. 5,905,146; 5,849,999; 5,859,311; 5,602,309; 5,952,169 & 5,772,997 (HER2 receptor).

Modulating or Inhibiting Expression. Using the gustatory receptor genes of the present invention, it is now possible to selectively modulate or inhibit specific DGR genes using antisense oligomers which specifically hybridize with the DNA or RNA encoding the DGR genes. One skilled in the art could so modulate or inhibit the expression of the DGR genes and detect for changes in taste response and behavior. Parallel studies may be conducted in other organisms by using the gustatory receptor genes and the methods of the present invention to identify the gustatory receptor genes in other organisms and then use antisense oligers to the gustatory receptor genes of those organisms. Methods for inhibiting expression of genes, especially genes coding for receptors, using antisense constructs, including generation of antisense sequences in situ are described, for example, in U.S. Pat. Nos. 5,856,099; 5,556,956; 5,716,846; 5,135,917 & 6,004,814.

Other methods that can be used to inhibit expression of an endogenous gene are applicable to the present invention. For example, formation of a triple helix at an essential region of a duplex gene serves this purpose. The triplex code, permitting design of the proper single stranded participant is also known in the art. (See Moser et al., (1987) Science 238, 645-650 and Cooney et al., (1988) Science 241, 456-459). Regions in the control sequences containing stretches of purine bases are particularly attractive targets. Triple helix formation along with photo-crosslinking is described, e.g., in Praseuth et al., (1988) Proc. Natl Acad. Sci. USA 85, 1349-1353.

Studying Behavior. Behavioral studies may help organize the gustatory systems in various organisms and may help explain the behavior of various organisms.

The tools and methodologies of the present invention may be used to study the influence of environmental conditions on eating behavior. For example, newly identified gustatory receptor genes may be used to study the effects of different preferences for a particular food source.

In one embodiment, modulation of gustatory receptor activity can be measured by the probosis extension response assay. When gustatory sensilla on either the labellum or the leg are stimulated with a sugar solution, the fly extends its mouthparts, in a behavior known as the proboscis extension response (PER). A variety of stimuli, including bitter compounds and high concentrations of salts, inhibit the PER when added to the sugar solution. The PER depends on the dose of the sugar solution, and the inhibition by other compounds is dose-dependent as well. The PER is simple to measure, and can be quantitated precisely. It has been characterized in great detail, initially in the classic experiments of Dethier on large flies such as the blowfly Phormia regina (Dethier (1955) Quart. Rev. Biol. 30, 348-371; Dethier, (1976) The Hungry Fly, Harvard Press).

In Drosophila, a PER has been shown to be elicited by sugar, and inhibited when NaCl is added to the sugar (Arora, (1987) Nature 330, 62-63; Rodrigues & Siddiqi (1978) Proc. Ind. Acad. Sci. 87B, 147-160; Tompkins et al., (1979) Proc. Natl. Acad. Sci. USA 76, 884-887).

In yet another embodiment, gustatory receptor activation assays may be based on the fact that flies demonstrate strong preferences when presented with two taste stimuli. Using a countercurrent behavioral paradigm in which flies make a series of binary choices between a sucrose medium either with or without quinine sulfate, it has been shown that flies preferentially distribute onto the medium without quinine (Tompkins et al., (1979) Proc. Natl. Acad. Sci. USA 76, 884-887), which tastes bitter to humans. Flies manifest preferences for different sugar solutions, as shown in an elegant paradigm in which animals are allowed to feed from the wells of a microtiter dish (Tanimura et al., (1982) J. Comp. Physiol. 147, 433-437). Wells of the dish contain agar, with alternate wells containing one of two sugars. Wells containing one sugar are marked with red dye; those containing the other sugar are marked with blue dye. After feeding in the dark, flies are classified according to the color of their abdomen (red, blue, or mixed), which provides a quantitative indication of their feeding preferences, which can be used as a measure for the activity of any particular gustatory receptor.

4. For Organism Detection, Monitoring and Control.

General Pest Management. The gustatory receptor genes identified herein and identified using the methods of the present invention may be used to identify compounds which may be used for pest management. It is especially desirable to utilize various aspects of the present invention for pest management related to crop protection.

The application of pheromones is now firmly established as a key component of pest management and control, especially within the framework of integrated pest management (IPM). An object of organism control is to modulate an organism's behavior or activity so as to reduce the irritation, sickness, or death of the host (e.g., a plant host), or to decrease the general health and proliferation of the organism.

For example, the propagation of a mouse population in a given area of actual or potential mice infestation may be prevented or inhibited by a bait containing an effective amount of a first compound which the mice prefer to eat, wherein such compounds could be combined with a second compound, such as a pheromone, which would attract the mice to the bait and would also be combined with a third compound which would have lethal effects on the mice. Thus, in a preferred embodiment, the mice would be attracted to the area by the odor of the second compound, enticed to eat a large amount of the bait because of the taste of the first compound and would die as a result of the presence of the third compound in the bait.

Compositions for attracting insects generally require some physical and/or chemical means for attracting the insects to a bait. In addition, the bait needs to be fully attractive to the taste of the insect so as to induce the attracted insect to ingest the bait. Finally, the bait must be taken in by the insect at a sufficient lethal dose before disgusting the insect in some way or producing a toxic reaction in the insect (see, for example, U.S. Pat. No. 4,855,133).

Insect Repellents and Insecticides. The present invention provides the tools and methodologies useful for identifying compounds which modulate insect behavior by exploiting the sensory capabilities of the target insect. For example, attempts have been made to describe and synthesize the complex interactions which underlie host-seeking behavior in mosquitoes. Using the methods and gustatory receptor genes of the present invention, it is possible to design specific compounds which target mosquito gustatory receptor genes. Thus, the present invention provides the ability to alter or to eliminate the orientation and feeding behaviors of mosquitoes and thereby have a positive impact on world health by controlling mosquito-borne diseases, such as malaria.

Mosquito gustatory receptor genes may be identified and/or targeted using various aspects of the present invention. For example, the gustatory receptor genes of the present invention may be used to design probes as discussed elsewhere herein for the identification and characterization of mosquito gustatory receptor genes. Alternatively, the algorithm of the present invention may be used to identify mosquito gustatory receptor genes in the genetic databases for mosquitoes. Once the mosquito gustatory receptor genes are identified, then various screening methods described elsewhere herein, such as the high throughput assays discussed elsewhere herein, may be used to identify synthetic and natural compounds which may modulate the behavior of the insect.

For general information on insect repellents, see, for example, U.S. Pat. No. 4,663,346.

Mating Enhancement and Disruption. The gustatory receptor genes identified herein and identified using the methods of the present invention may be used to identify compounds which interfere with the orientation and mating of a wide range of organisms, including insects. Thus, the present invention enables the identification of compositions which disrupt insect mating by selective inhibition of specific receptor genes involved in mating attraction (see, e.g., U.S. Pat. No. 5,064,820).

Animal Repellants. The gustatory receptor genes identified herein and identified using the methods of the present invention may be used to identify compounds which may be used as animal repellants. Such compositions may be used to repel both predatory and non-predatory animals (see, e.g., U.S. Pat. No. 4,668,455).

5. Organism Attraction.

Insect Attractants. The gustatory receptor genes identified herein and identified using the methods of the present invention may be used to identify compounds which attract specific insects to a particular location (see, e.g., U.S. Pat. Nos. 4,880,624 & 4,851,218).

For example, aspects of the present invention may to used in various methods which reduce or eliminate the levels of particular insect pests by selective attraction of a particular insect or pest, such as mosquitoes and tsetse flies. As a particular example, insect traps can be created wherein the taste of a compound selectively attracts a particular insect, like the tsetse fly, and the insect so attracted dies in the trap. Once in the trap, the attraction is maintained by stimulation of a particular gustatory receptor of the invention. In this way, the population of tsetse flies may be reduced or eliminated in a particular area.

The identified compositions which selectively attract and maintain the attraction by stimulation of gustatory receptors may also be combined with an insecticide, for example as an insect bait in microencapsulated form. Alternatively, or in addition, the insect attractant composition may be placed inside an insect trap, or in the vicinity of the entrance to an insect trap.

In addition to killing insects, the trapping of insects is often very important for estimating or calculating how many insects of a particular type are feeding within a specific area. Such estimates are used to determine where and when insecticide spraying should be commenced and terminated.

Insect traps which may be used are, for example, those as described in U.S. Pat. No. 5,713,153. Specific examples of insect traps include, but are not limited to, the Gypsy Moth Delta Trap®, Boll Weevil Scout Trap®, Jackson trap, Japanese beetle trap, McPhail trap, Pherocon IC trap, Pherocon II trap, Perocon AM trap and Trogo trap.

Kairomones may be used as an attractancy for the enhancement of the pollination of selected plant species.

Attractant compositions which demonstrate biological activity toward one sex which is greater than toward the opposite sex may be useful in trapping one sex of a specific organism over another. For example, a composition may be a highly effective attractant for male apple ermine moths (Yponomeuta malinellus (Zeller)) and not so effective an attractant for female apple ermine moths. By attracting and maintaining the attraction of adult males to field traps, the composition provides a means for detecting, monitoring, and controlling this agricultural pest (see, e.g., U.S. Pat. No. 5,380,524).

Attracting Predators and Parasitoids. The gustatory receptor genes of the present invention and the gustatory receptor genes identified using the methods of the present invention may also be used to identify chemicals which attract and maintain the attraction of various predators and parasitoids. Attracting the predators and parasitoids which attack certain pests offers an alternative method of pest management.

Animal Attractants. The gustatory receptor genes identified herein and those identified by the methods of the present invention may be used to identify chemicals which attract household domesticated animals. For example, a pheromone-containing litter preparation may attract the animals and absorb liquids and liquid-containing waste released by the attracted animal (see, e.g., U.S. Pat. No. 5,415,131).

6. Industrial Applications. The gustatory receptor genes identified by the methods of the present invention may be used for a number of different industrial applications including, but not limited to the following:

(a) Identification of appetite suppressant compounds and using same to suppress and/or control appetite.

(b) As Biosensors.

(1) Explosive and drug detectors. The detectors may be synthetic, such as biologically-inspired robotic sensors, or biological sensors, such as insects which are especially sensitive to certain tastes.

(2) Population of gustatory receptor genes expressed in cell culture. Gustatory receptor genes can be introduced into a cell line and the transformed cells maintained in culture through multiple generations. By creating specific cell lines which express multiple gustatory genes at once, it would be possible to use such cell cultures to investigate how compounds interact with taste receptor genes. Thus, the present invention provides methods for identifying taste fingerprints, wherein such methods include contacting a series of cells containing and expressing known gustatory receptor genes with a desired sample, and determining the type and quantity of the gustatory receptor ligands present in the sample (see, e.g., U.S. Pat. No. 5,993,778). As discussed elsewhere herein, the interaction of substances with the receptors can be identified using appropriate labels, such as those provided by luciferase, the jellyfish green fluorescent protein (GFP) or β-galactosidase.

(3) Biochip Arrays. As discussed elsewhere herein, biochip arrays of gustatory receptor genes can be generated. The arrays may be used to detect gustatory receptor ligands via an appropriate marker or via a chemical or electrical signal. Arrays may be designed for specific purposes, such as, but not limited to, detecting perfumes, explosives, drugs, pollutants, and toxins.

(c) Training organisms to conduct certain tasks. Examples include, but are not limited to, orienting or reorienting the behavior of worker bees of a rearing colony by incorporating a composition which includes one or more pheromones which elicits particular bee behavior towards the larvae. Thus, the beekeeper may orient or reorient the bees towards a particular activity such as, but not limited to, inducing improved acceptance of the larvae at the beginning of rearing, to increase the production of royal jelly, regulate the feeding of the larvae as to favor the development of queen bees, etc. (see, e.g., U.S. Pat. No. 5,695,383).

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Candidate Taste Receptor Genes

With approximately 100% of the Drosophila genome sequenced, the Drosophila gustatory receptor genes have been sequenced. A multi-step strategy was developed to identify taste receptor genes from the genomic database. First, a computer algorithm was designed to search the Drosophila genomic sequence for open reading frames (ORFs) from candidate taste receptor genes. Second, RT-PCR was used to determine if transcripts from any of these ORFs identified through this approach were expressed in specific tissues and organs, including taste tissue deficient in chemosensory neurons.

### Example 2

### Algorithm for Identification of G Protein-Coupled Receptors (GPCR) Genes

A computer algorithm that seeks proteins with particular structural properties, as opposed to proteins with particular sequences, identified a large family of candidate gustatory receptors from the Drosophila genomic database (Clyne et al., (1999) Neuron 22, 327-338 incorporated herein in its entirety). The algorithm examines the physicochemical properties of the amino acids in an open reading frame (ORF) and then uses a non-parametric discriminant function to identify ORFs likely to encode multitransmembrane domain proteins.

The algorithm used to identify G protein-coupled receptors (GPCR) genes used statistical characterization of amino acid physico-chemical profiles in combination with a non-parametric discriminant function. The key approach is to use the information in the interplay between the local structure (transmembrane alpha helix) and the global structure (repeated multiple domains) and characterize this information with concise statistical variables.

The algorithm was trained on a set of one-hundred putative GPCR sequences from the GPCR database (GPCRDB) at http://swift.embl-heidelberg.de/7tm and a set of one-hundred random proteins selected from the SWISSPROT database (this training set was later expanded, but that version was not used for the genes reported in this paper). In the first step, three sets of descriptors were used to summarize the physico-chemical profiles of the sequences. These were GES scale of hydropathy (Engelman et al., (1986) Annu. Rev. Biophys. Biophys. Chem. 15, 321-353), polarity (Brown, (1991) Molecular Biology Labfax, Academic Press), and amino acid usage frequency. For the first two of these measurements, a sliding window profile was employed (White, (1994) Membrane Protein Structure, Oxford University Press) using a kernel of 15 amino acid constant function convoluted with a 16 amino acid Gaussian function.

These profiles were then summarized with three statistics; the periodicity (characterizing the quasi-periodic presence of the transmembrane domain), average derivative (characterizing the abrupt change between the transmembrane domain and non-transmembrane domain), and the variance of the derivative (also characterizing the abrupt change). GES periodicity, variance of polarity derivative, polarity periodicity and amino acid frequency were used as the four variables and each sequence was therefore characterized by four variables. These four variables were used in a non-parametric linear discriminant function that was then optimized to separate the known GPCRs from random proteins in the training set. The same linear discriminant function with the scores derived from the training set was then used to screen the genomic database for candidate genes.

The candidate sequences were given significance values by an odds ratio of the GPCRs and non-GPCRs computed using the observed empirical distribution of the training set. More detailed information about the algorithm is available at http://www.neuron.org/cgi/content/full/22/2/327/dcl.

The computational screens used the genomic sequence data obtained by FTP from the Berkeley Drosophila Genome Project (BDGP, http://www.fruitfly.org, version 6/98). First, the ORFs of 300 bases or longer in all six frames were identified. Next, a program written to identify GPCRs statistically by their physico-chemical profile was used to screen for candidate ORFs as described above. The number of possible candidates was reduced by comparing them to Drosophila codon usage tables (http://flybase.bio.indiana.edu, version 10). Candidate ORFs whose codon usage differed at a significance level of 0.0005 by the chi-square statistic were discarded from the candidate set. Using these screening steps, thirty-four candidate ORFs were obtained.

### Example 3

### Further Analysis of Genes Identified by the Algorithm

Further analysis of genes identified by this algorithm revealed one gene that led to the definition of a distinct large DGR family of membrane proteins. Forty-three members of this family have been identified in the complete Drosophila genome. If the sequenced part of the genome is representative, then extrapolation suggests that the entire genome would encode on the order of 75 DGR proteins, a figure comparable to previous estimates of 100 candidate Drosophila odorant receptors (DOR), as described in Clyne et al., (1999) Neuron 22, 327-338).

This previously unidentified family of proteins shows no sequence similarities to odorant receptors or to other known proteins. This family of proteins has been designated the gustatory receptor (GR) family, with each individual gene named according to its cytogenetic location in the genome. Thus, the GR59D.1 and GR59D.2 genes, which was abbreviated here as 59D.1 and 59D.2, refer to two family members located in cytogenetic region 59D on the second chromosome. This designation of location, however, does not reflect additions to the Drosophila genome subsequent to the discovery of the gustatory receptor genes.

The first exon of 23A.1b (FIG. 1) was identified by the computer algorithm described in Clyne et al., (1999) Neuron 22, 327-338, as described in Example 2, above.

Examination of the genomic DNA surrounding the first exon of 23A.1b identified other exons, and the genomic structure of this gene was determined with RT-PCR.

Using the sequence of this gene, an extensive series of tBLASTn searches of the Berkeley Drosophila Genome Project (BDGP) sequence database (available at http://www.fruitfly.org) was performed, which identified ORFs of thirty-eight other genes of the GR family. The full sequences of these genes were identified by an analysis of the genomic DNA flanking these ORFs as described in Clyne et al., (1999) Neuron 22, 327-338 using the Drosophila intron-exon consensus splice sequences and RT-PCR analysis. The thirty-nine genes encode a total of forty-three proteins.

The National Center for Biotechnology Information (NCBI) accession number of the BDGP genomic clone on which each transcript is found and the sequence range in the genomic clone for the predicted coding region are given as follows for each GR transcript shown in FIG. 1 (NCBI and BDGP data are as of Oct. 16, 1999): transcript GR21D.1, accession number AC004420, range 34784-33509; GR22B.1, AC003945, 31740-30551; GR23A.1a, AC005558, 108490-106118; GR23A.1b, AC005558, 107351-106118; GR32D.1, AC005115, 19779-21141; GR39D.1, AC007208, 62553-64348; GR39D.2a, AC005130, 9170-16119; GR39D.2b, AC005130, 10410-16119; GR39D.2c, AC005130, 12989-16119; GR39D.2d, AC005130, 14750-16119; GR43C.1, AC005452, 50105-51583; GR47A.1, AC007352, 114644-115920; GR58A.1, AC004368, 62323-61087; GR58A.2, AC004368, 62511-63791; GR58A.3, AC004368, 65521-64229; GR59D.1, AC006245, 68825-70050; GR59D.2, AC006245, 70261-71505; GR59E.1, AC005639, 30167D31539; and GR59E.2, AC005639, 30036-28714.

Accession numbers for the other genes are as follows (data are as of Jan. 5, 2000) (complete sequences are available for the first four and only partial sequences are available for the remaining genes; LU, location unknown): transcript GR1F.1, accession number AL035632, range 7301-8711; GR47F.1, AC005653, 42838-44204; GR68D.1, AC006492, 46040-44916; GR77E.1, AC006490, 104929-103117; GR28A.1, AC008354, 66711-66973; GR57B.1, AC007837, 102661-103185; GR65C.1, AC004251, 23136-24215; GR93F.1, AC012873, 35043-35228; GR93F.2, AC012892, 2781-2650; GR93F.3, AC012892, 4271-4143; GR93F.4, AC012892, 6482-5559; GR94E.1, AC008200, 72472-72308; GR97D.1, AC007984, 121300-121977; GR98B.1, AC007817, 45506-46916; GR98B.2, AC007817, 10695-10784; GR98B.3, AC007817, 45189-45284; GR98B.4, AC007817, 39658-39765; GRLU.1, AC017438, 22141-21398; GRLU.2, AC017138, 10997-11122; GRLU.3, AC015395, 43210-43612; GRLU.4, BACR28P1-T7, 28-129; GRLU.5, BACR28P1-T7,388-734; GRLU.6, BACR06I03-T7, 1028-48; and GRLU.7, AC012799, 8212-8123.

### Example 4

### Sequence Analysis of DGR Genes

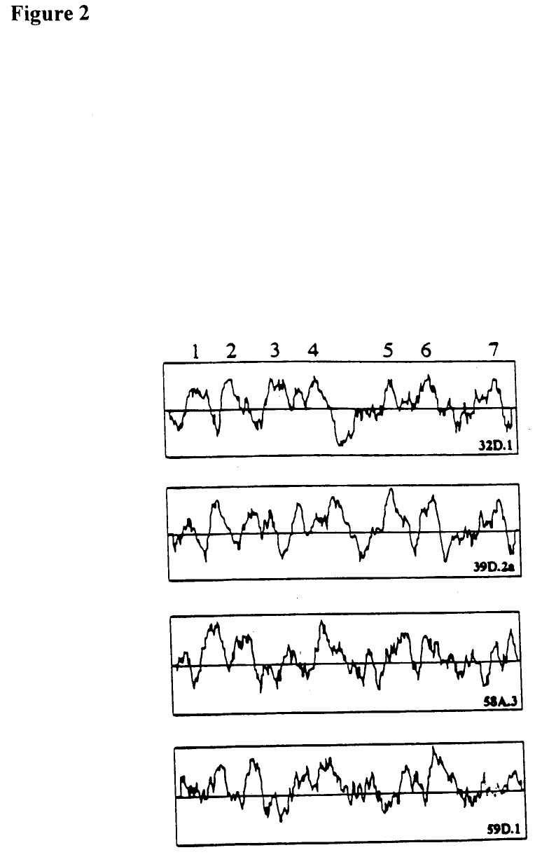

The amino acid sequences of nineteen members of the GR family indicate the high degree of sequence divergence (FIG. 1). Sequence alignment revealed only one residue conserved among all members of the family shown and only 24 residues conserved among more than half of the genes shown. Fifteen of these conserved residues lie in the vicinity of the COOH-terminus. Amino acid identity between individual genes ranged from a maximum of 34% to less than 10%. By contrast, other features of the gene family show substantial conservation. The positions of a number of introns are conserved (FIG. 1), suggesting that the family originated from a common ancestral gene. Overall sequence length, ˜380 amino acids, is another common feature. All of the genes encode approximately seven predicted transmembrane domains, a feature characteristic of G protein-coupled receptors (GPCRs) (FIG. 2).

The GR proteins were identified as GPCRs when the algorithm was modified to distinguish previously described GPCRs from ion channels. The algorithm was set to positively identify 95% of previously described GPCRs, with 4.3% false positives. Most ion channels have six transmembrane domains.

The genes are widely dispersed in the genome, but at the same time, many are found in clusters. The two largest clusters each contain four genes; there are also several clusters of two or three genes. Genes within these clusters are closely spaced, with intergenic distances ranging from 150 to 450 base pairs (bp) in all cases for which the data were available. There is no rule specifying the orientation of genes within clusters, unlike the case of the Drosophila odorant receptors, in which genes within a cluster are in the same orientation in all clusters examined (Clyne et al., (1999) Neuron 22, 327-338).

An unusual form of alternative splicing occurs in at least two chromosomal locations. Four large exons in cytogenetic region 39D each contain sequences specifying six predicted transmembrane domains, followed by three small exons that together specify a putative seventh transmembrane domain and the COOH-terminus (FIG. 3). Reverse transcription-polymerase chain reaction (RT-PCR) analysis revealed that each of the four large exons is spliced to the smaller exons, thereby generating four predicted seven transmembrane domain proteins. These four proteins are thus distinct through the first six transmembrane domains and identical in the seventh and in the COOH-terminal sequences. Likewise, in cytogenetic region 23A, there are two large exons, each of which specifies six transmembrane domains and is spliced to two small exons that together encode a seventh transmembrane domain and the COOH-terminus (FIG. 3). Thus, the gene in region 23A encodes two related proteins. This pattern of splicing, in which alternative large 5′ exons encoding most of the protein are joined to common short 3′ exons encoding only a small portion of the protein, is unusual among genes encoding GPCRs and proteins in general. This pattern of splicing provides a mechanism at a single locus for generating products that exhibit a pattern observed for this family in general: extreme diversity among all sequences of the proteins except in a small region in the vicinity of the COOH-terminus.

### Example 5

### Identification of Gustatory Receptor Genes Using RT-PCR

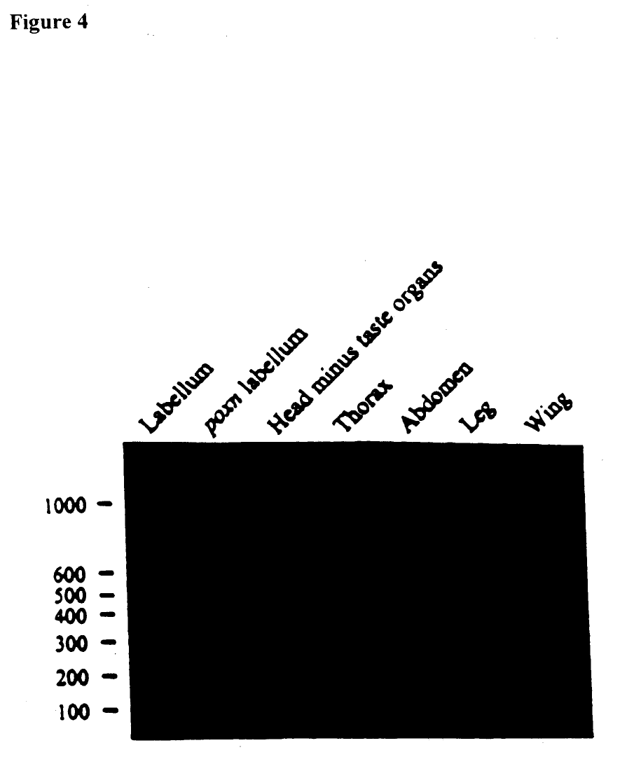

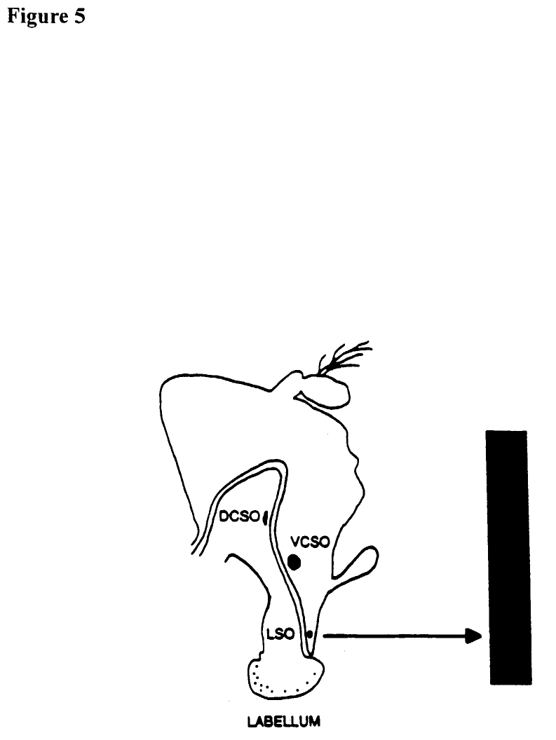

To assess the tissue specificity of expression, RT-PCR with primers that span introns in the coding regions was performed. Of the 19 transcripts tested, 18 were expressed in the labellum (FIG. 4 and Table 1), the major gustatory organ of the fly (Falk et al., (1976) J. Morphol. 150, 327; Dethier, (1976) The Hungry Fly, Harvard University Press; Stocker, (1994) Cell Tissue Res. 275, 3; Nayak & Singh, (1983) Int. J. Insect Morphol. Embryol. 12, 273. Moreover, for most of these genes, expression was labellum-specific in that only 1 of the 19 yielded amplification products from heads depleted of taste organs and only 2 showed expression in the thorax, which contains the thoracic nervous system but no characterized taste sensilla. Likewise, expression in several other tissues, including the abdomen, wings, and legs, was limited to a small fraction of genes (Table 1).

For preparation of RNA, individual flies were frozen in liquid nitrogen, and labella were dissected. On average, 50 labella were used for RNA preparation. Total RNA was prepared as described elsewhere (McKenna et al., (1994) J. Biol. Chem. 269, 16340-16347). The RNA was treated with DNaseI (Gibco-BRL) for thirty minutes at 37° C., phenol/chloroform extracted, and precipitated. The entire RNA preparation was used for oligo dT-primed cDNA synthesis using Superscript II Reverse Transcriptase (Gibco-BRL) according to the manufacturer's directions. PCR was performed using Taq polymerase (Sigma) under standard cycling conditions, with an annealing temperature of 60° C., gene-specific primer concentration of 1 pM, and magnesium concentration of 2.5 mM. For all genes, primer pairs which span introns were used in order to distinguish PCR bands amplified from cDNA from those amplified from any remaining genomic DNA.

### Example 6

### Tissue Specificity of GR Gene Expression

To further analyze the tissue specificity of GR expression, a microdissection experiment was performed in which the labral sense organ (LSO) (Stocker, (1994) Cell Tissue Res. 275, 3; Nayak & Singh, (1983) Int. J. Insect Morphol. Embryol. 12, 273), a small taste organ that lines the pharynx, was surgically excised from each of fifty animals. The LSO consists of a very limited number of cells and is highly enriched in taste neurons; it does not, for example, contain muscle cells. By RT-PCR amplification, the expression of seven GR transcripts in this taste organ was detected (FIG. 5). These results indicate that expression of the GR family extends to include at least one additional taste organ besides the labellum The data are also fully consistent with the notion that the GR genes are expressed in taste neurons.

To confirm the gene expression in taste receptor neurons, a Drosophila mutant, pox-neuro 70 (poxn 70) was used, in which chemosensory bristles are transformed into mechanosensory bristles (Awasaki & Kimura, (1997) J. Neurobiol. 32, 707; Dambly-Chaudiere et al., (1992) Cell 69, 159; Nottebohm et al., (1994) Neuron 12, 25; Nottebohm et al., (1992) Nature 359, 829).

Specifically, in poxn 70, which behaves as a null mutation with respect to adult chemosensory organs, chemosensory bristles are transformed into mechanosensory bristles with respect to various morphological and developmental criteria. In particular, most chemosensory bristles in wild-type Drosophila are innervated by five neurons: four 58 chemosensory neurons and one mechanosensory neuron. In contrast, wild-type mechanosensory bristles contain a single mechanosensory neuron.

In chemosensory bristles transformed to mechanosensory bristles by poxn 70 (Awasaki & Kimura, (1997) J. Neurobiol. 32, 707), the number of neurons is reduced from five to one. We predicted that if the GR family is in fact expressed in the chemosensory neurons of taste sensilla, their expression would likely be eliminated in the poxn 70 mutant. Consistent with this prediction, eighteen of nineteen GR transcripts examined were not expressed in the labellum of the poxn 70 mutant (Table 1 and FIG. 4). RT-PCR was performed from RNA extracted from the indicated tissues (see description for FIG. 4). All primer pairs spanned introns. Positive controls are described in FIG. 4. These results indicate that the GR gene family is expressed in labellar chemosensory neurons.

TABLE 1
Tissue-specific expression of GR genes.

| | | | Head | | | | |
|---|---|---|---|---|---|---|---|
| | | | minus | | Ab- | | |
| | | poxn | taste | | do- | | |
| Gene | Labellum | Labellum | organs | Thorax | men | Leg | Wing |
| 21D.1 | + | − | − | − | − | − | − |
| 22B.1 | + | − | − | − | + | + | + |
| 23A.1a | + | − | − | − | − | − | − |
| 23A.1b | + | − | − | − | − | − | − |
| 32D.1 | + | − | − | − | − | − | − |
| 39D.1 | + | − | − | − | − | − | − |
| 39D.2a | + | − | − | − | − | − | − |
| 39D.2b | + | − | − | − | − | − | − |
| 39D.2c | + | + | − | + | + | − | − |
| 39D.2d | + | − | − | + | − | − | + |
| 43C.1 | + | − | + | − | + | + | + |
| 47A.1 | + | − | − | − | − | − | − |
| 58A.1 | + | − | − | − | − | − | − |
| 58A.2 | + | − | − | + | − | − | − |
| 58A.3 | + | − | − | − | − | − | − |
| 59D.1 | + | − | − | − | − | − | − |
| 59D.2 | + | − | − | − | − | − | − |
| 59E.1 | − | − | − | − | + | − | + |
| 59E.2 | + | − | − | − | − | − | − |

### Example 7

### Receptor Diversity

The large size of this protein family likely reflects the diversity of compounds that flies can detect. The extreme diversity of these receptors may not only reflect diversity among the ligands that they bind, but also diversity in the signal transduction components with which they interact. For example, the lack of conserved intracellular regions suggests the possibility that, during the evolution of this sensory modality, multiple G proteins arose, each interacting with a different subset of receptors. Finally, it seems likely that the Drosophila genome encodes taste receptors in addition to those of the GR family.

Although applicants have detected expression in the labellum and the LSO, few if any family members are expressed in the leg or wing chemosensory hairs (Table 1), some of which are morphologically similar to labellar taste hairs (Stocker, (1994) Cell Tissue Res. 275, 3). The Drosophila olfactory system also contains more than one organ, the antenna and maxillary palp, which respond to all, or nearly all, of the same odorants and which derive from the same imaginal discs (Carlson, (1996) Trends Genet. 12, 175). However, most individual members of the DOR gene family are expressed in one or the other but not in both olfactory organs (Clyne, (1999) Neuron. 22, 327; Vosshall et al., (1999) Cell 96, 725). Perhaps the distinction among taste receptor genes is even more extreme in the gustatory system, whose organs derive from different imaginal discs. For example, the legs may express a completely distinct family of genes or a subfamily whose similarities to the present family are sufficiently tenuous as to place it slightly beyond the boundaries that define the GR family.

### Example 8

### Transgenic Drosophila

P element mediated germline transformation of Drosophila can be carried out as previously described (Rubin & Spradling, (1982) Science 218, 348-353). Drosophila embryos are isolated and microinjected with P element expression constructs as previously described (Karess & Rubin, (1984) Cell 38, 135-146) containing a particular DGR nucleotide sequence, at 0.5 mg/ml together with a helper plasmid at 0.1 mg/ml.

Non-transformed (Generation 0 or Go) injected adults are individually back crossed to the recipient strain and the Gi progeny screened for the w+transformation marker (Klemenz et al., (1987) Nucleic Acids Res. 10, 3947-3959). Transformed lines homozygous for the transgene are established from orange eyed Gi flies as previously described (Klemenz et al., (1987) Nucleic Acids Res. 10, 3947-3959).

A line of Drosophila in which the 39D.2c gene can be over-expressed is constructed as described above. The 39D.2c coding sequences are joined to an upstream activating sequence (UAS) and introduced by P element-mediated germline transformation into Drosophila. A yeast GAL4 transcription factor gene, coupled to a heat shock promoter, is then crossed into the transgenic line. As expected, heat shock of this line results in induction of 39D.2c expression. The heat shock-induced expression of GAL4, also results in binding of GAL4 to the UAS, and subsequent induction of 39D.2c expression. This transgenic line of Drosophila, and three other transgenic lines containing other DGR genes, can be tested for elevated responses to any of fifty different tastes. Elevated response to any particular taste is indicative of an ligand which binds and activates the over-expressed receptor (see, e.g., Zhao & Firestein, (1998) Science 279, 237-242).

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety. The results of the experiments disclosed herein have been published in the journal Science (2000) 287, 1830-1834, this article herein incorporated by reference in its entirety.

We claim:

1. An isolated nucleic acid molecule selected from the group consisting of:
a) an isolated nucleic acid molecule that encodes the amino acid sequence of a Drosophila Gustatory Receptor protein;
b) an isolated nucleic acid molecule that encodes a protein fragment of at least 6 amino acids of a Drosophila Gustatory Receptor protein; and
c) an isolated nucleic acid molecule which hybridizes to a nucleic acid molecule comprising a nucleotide sequence encoding a Drosophila Gustatory Receptor protein under conditions of sufficient stringency to produce a clear signal.

2. The isolated nucleic acid molecule of claim 1 wherein the nucleic acid comprises at least one exon-intron boundary located in a position selected from the group consisting of:
a) the nucleotides encoding the amino acids which comprise the third extracellular loop of a Drosophila Gustatory Receptor protein; and
b) the nucleotides encoding the amino acids which comprise the seventh transmembrane domain of a Drosophila Gustatory Receptor protein.

3. The isolated nucleic acid molecule of claim 1, wherein the nucleic acid molecule is selected from the group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 90 and 91.

4. The isolated nucleic acid molecule of any one of claims 1-3, wherein said nucleic acid molecule is operably linked to one or more expression control elements.

5. A vector comprising an isolated nucleic acid molecule of any one of claims 1-3.

6. A host cell transformed to contain the nucleic acid molecule of any one of claims 1-3.

7. A host cell comprising a vector of claim 5.

8. A host cell of claim 7, wherein said host is selected from the group consisting of prokaryotic hosts and eukaryotic hosts.

9. A method for producing a protein or protein fragment comprising the step of culturing a host cell transformed with the nucleic acid molecule of any one of claims 1-3 under conditions in which the protein or protein fragment encoded by said nucleic acid molecule is expressed.

10. The method of claim 9, wherein said host cell is selected from the group consisting of prokaryotic hosts and eukaryotic hosts.

11. An isolated protein or protein fragment produced by the method of claim 10.

12. An isolated protein or protein fragment selected from the group consisting of:
a) an isolated protein comprising one of the amino acid sequences depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92;
b) an isolated protein fragment comprising at least six amino acids of any of the sequences depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92;
c) an isolated protein comprising conservative amino acid substitutions of any of the sequences depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92; and
d) naturally occurring amino acid sequence variants of any of the sequences depicted in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90 and 92.

13. The isolated protein or protein fragment of claim 12 wherein the protein or protein fragment of a Drosophila Gustatory Receptor protein has at least one of the following conserved amino acids selected from the group consisting of:
a) Serine in the amino terminal domain;
b) Phenylalanine in the first transmembrane domain;
c) Arginine in the first extracellular loop;
d) Leucine in the fourth transmembrane domain;
e) Leucine in the third transmembrane domain;
f) Glycine in the fifth transmembrane domain;
g) Tyrosine in the fifth transmembrane domain;
h) Leucine in the third extracellular loop;
i) Phenylalanine in the third extracellular loop;
j) Alanine in the seventh transmembrane domain;
k) Glycine in the seventh transmembrane domain;
l) Leucine in the seventh transmembrane domain;
m) Aspartate in the seventh transmembrane domain;
n) Alanine in the seventh transmembrane domain;
o) Threonine in the seventh transmembrane domain;
p) Tyrosine in the seventh transmembrane domain;
q) Valine in the seventh transmembrane domain;
r) Glutamine in the carboxy terminal domain; and
s) Phenylalanine in the carboxy terminal domain.

14. An isolated antibody that binds to a polypeptide of claim 11, 12 or 13.

15. The antibody of claim 14 wherein said antibody is a monoclonal or polyclonal antibody.

16. A method of identifying an agent which modulates the expression of a protein or protein fragment of claim 11, 12 or 13 comprising the steps of:
a) exposing cells which express the protein or protein fragment to the agent; and
b) determining whether the agent modulates expression of said protein or protein fragment, thereby identifying an agent which modulates the expression of a protein or protein fragment of claim 11, 12 or 13.

17. A method of identifying an agent which modulates the activity of a protein or protein fragment of claim 11, 12 or 13 comprising the steps of:
a) exposing cells which express the protein or protein fragment to the agent; and
b) determining whether the agent modulates the activity of said protein or protein fragment, thereby identifying an agent which modulates the activity of a protein or protein fragment of claim 11, 12 or 13.

18. The method of claim 17, wherein the agent modulates at least one activity of the protein or protein fragment.

19. A method of identifying an agent which modulates the transcription of the nucleic acid molecule of any one of claims 1-3 comprising the steps of:
a) exposing cells which transcribe the nucleic acid to the agent; and
b) determining whether the agent modulates transcription of said nucleic acid, thereby identifying an agent which modulates the transcription of the nucleic acid molecule of any one of claims 1-3.

20. A method of identifying binding partners for a protein or protein fragment of either claim 11, 12 or 13 comprising the steps of:
a) exposing said protein or protein fragment to a potential binding partner; and
b) determining if the potential binding partner binds to said protein or protein fragment, thereby identifying binding partners for the protein or protein fragment.

21. A method of modulating the expression of a nucleic acid encoding a protein or protein fragment of claim 11, 12 or 13 comprising administering an effective amount of an agent which modulates the expression of a nucleic acid encoding the protein or protein fragment.

22. A method of modulating at least one activity of a protein or protein fragment of claim 11, 12 or 13 comprising the step of administering an effective amount of an agent which modulates at least one activity of the protein or protein fragment.

23. A method of identifying novel gustatory receptor genes comprising the steps of:
a) selecting candidate gustatory receptor genes by screening a nucleic acid database using an algorithm trained to identify seven transmembrane receptors genes;
b) screening said selected candidate gustatory receptor genes by identifying nucleic acid sequences with conserved amino acid residues and intron-exon boundaries common to gustatory receptors, and having open reading frames of sufficient size so as to encode a seven transmembrane receptor; and
c) identifying the novel gustatory receptor genes and measuring the expression of gustatory receptor genes wherein the detection of expression confirms said candidate gustatory gene as an gustatory gene.

24. A method of identifying novel gustatory receptor genes comprising the steps of:
a) selecting candidate gustatory receptor genes by screening a nucleic acid database for nucleic acid sequences with sufficient homology to at least one known gustatory receptor gene;
b) screening said selected candidate gustatory receptor genes by identifying nucleic acids with conserved amino acid residues and intron-exon boundaries common to gustatory receptors, and having open reading frames of sufficient size so as to encode a seven transmembrane receptor; and
c) identifying the novel gustatory receptor genes and measuring the expression of gustatory receptor genes wherein the detection of expression confirms said candidate gustatory gene as an gustatory gene.

25. A transgenic insect modified to contain a nucleic acid molecule of any of claims 1-3.

26. The transgenic insect of claim 25, wherein the nucleic acid molecule contains a mutation that alters expression of the encoded protein.
